SEQUENCE LISTING (51 sequences)

SEQ ID NO: 1 - protein, 656 aa, synthetic construct
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQSSFRQDHPSSMGVYGQESGGFSGPGENRSMSGPDNRGRGRGGFDRGGMSRGGRGGGRGGMGSAGERGGFNKPGGPMDEGPDLDLGPPVDPDEDSDNSAIYVQGLNDSVTLDDLADFFKQCGVVKMNKRTGQPMIHIYLDKETGKPKGDATVSYEDPPTAKAAVEWFDGKDFQGSKLKVSLARKKPPMNSMRGGLPPREGRGMPPPLRGGPGGPGGPGGPMGRMGGRGGDRGGFPPRGPRGSRGNPSGGGNVQHRAGDWQCPNPGCGNQNFAWRTECNQCKAPKPEGFLPPPFPPPGGDRGRGGPGGMRGGRGGLMDRGGPGGMFRGGRGGDRGGFRGGRGMDRGGFGGGRRGGPGGPPGPLMEQMGGRRGGRGGPGKMDKGEHRQERRDRPY

SEQ ID NO: 2 - protein, 449 aa, synthetic construct
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 3 - protein, 17 aa, synthetic construct
SSSYGQQSEKPYQCDFK

SEQ ID NO: 4 - protein, 9 aa, synthetic construct
SSYGQQSEK

SEQ ID NO: 5 - protein, 10 aa, synthetic construct
SSSYGQQSEK

SEQ ID NO: 6 - protein, 9 aa, synthetic construct
YGQQSEKPY

SEQ ID NO: 7 - protein, 9 aa, synthetic construct
SEKPYQCDF

SEQ ID NO: 8 - protein, 10 aa, synthetic construct
SYGQQSEKPY

SEQ ID NO: 9 - protein, 15 aa, synthetic construct
CALSEMQGGSEKLVF

SEQ ID NO: 10 - protein, 15 aa, synthetic construct
CASSTSSGSPDTQYF

SEQ ID NO: 11 - protein, 136 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEMQGGSEKLVFGKGTKLTVNP

SEQ ID NO: 12 - protein, 133 aa, synthetic construct
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSTSSGSPDTQYFGPGTRLTVL

SEQ ID NO: 13 - protein, 14 aa, synthetic construct
CAGQGASGTYKYIF

SEQ ID NO: 14 - protein, 12 aa, synthetic construct
CASSPGTSDEFF

SEQ ID NO: 15 - protein, 130 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQGASGTYKYIFGTGTRLKVLA

SEQ ID NO: 16 - protein, 131 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGTSDEFFGPGTRLTVL

SEQ ID NO: 17 - protein, 13 aa, synthetic construct
CAMGGGSNYKLTF

SEQ ID NO: 18 - protein, 15 aa, synthetic construct
CASSPPGQTNYGYTF

SEQ ID NO: 19 - protein, 133 aa, synthetic construct
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGGGSNYKLTFGKGTLLTVNP

SEQ ID NO: 20 - protein, 133 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSPPGQTNYGYTFGSGTRLTVV

SEQ ID NO: 21 - protein, 141 aa, Homo sapiens
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 137 aa, synthetic construct
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 23 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 24 - protein, 179 aa, Homo sapiens
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 25 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 26 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 27 - protein, 27 aa, synthetic construct
RAKRSGSGATNFSLLKQAGDVEENPGP

SEQ ID NO: 28 - protein, 365 aa, synthetic construct
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQSEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 29 - protein, 7 aa, synthetic construct
TRDTTYY

SEQ ID NO: 30 - protein, 8 aa, synthetic construct
RNSFDEQN

SEQ ID NO: 31 - protein, 5 aa, synthetic construct
SGDLS

SEQ ID NO: 32 - protein, 6 aa, synthetic construct
YYNGEE

SEQ ID NO: 33 - protein, 5 aa, synthetic construct
SIFNT

SEQ ID NO: 34 - protein, 7 aa, synthetic construct
LYKAGEL

SEQ ID NO: 35 - protein, 5 aa, synthetic construct
SGHNS

SEQ ID NO: 36 - protein, 6 aa, synthetic construct
FNNNVP

SEQ ID NO: 37 - protein, 6 aa, synthetic construct
NSAFQY

SEQ ID NO: 38 - protein, 6 aa, synthetic construct
TYSSGN

SEQ ID NO: 39 - protein, 6 aa, synthetic construct
VSPFSN

SEQ ID NO: 40 - protein, 7 aa, synthetic construct
MTFSENT

SEQ ID NO: 41 - protein, 16 aa, synthetic construct
CVVSAARGSTLGRLYF

SEQ ID NO: 42 - protein, 5 aa, synthetic construct
SNHLY

SEQ ID NO: 43 - protein, 6 aa, synthetic construct
FYNNEI

SEQ ID NO: 44 - protein, 17 aa, synthetic construct
CASSLGQSSSYNSPLHF

SEQ ID NO: 45 - protein, 136 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAARGSTLGRLYFGRGTQLTVWP

SEQ ID NO: 46 - protein, 136 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSLGQSSSYNSPLHFGNGTRLTVT

SEQ ID NO: 47 - protein, 9 aa, synthetic construct
VTLTHPITK

SEQ ID NO: 48 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 49 - protein, 210 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 50 - protein, 458 aa, synthetic construct
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 51 - protein, 9 aa, synthetic construct
AVDLSHFLK

(19) United States
(12) Patent Application Publication
(10) Pub. No.: US 2025/0262244 A1
(43) Pub. Date: Aug. 21, 2025

(54) T CELL RECEPTORS TARGETING EWSR1-WT1 FUSION PROTEIN AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Christopher A. KLEBANOFF, New York, NY (US); Smita S. CHANDRAN, Long Island City, NY (US); Lauren B. BANKS, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/191,892

(22) Filed: Apr. 28, 2025

Related U.S. Application Data
(63) Continuation of application No. PCT/US2023/078139, filed on Oct. 30, 2023.
(60) Provisional application No. 63/381,328, filed on Oct. 28, 2022.

(51) Int. Cl. A61K 35/17; A61K 40/11; A61K 40/15; A61K 40/32; A61K 40/42; A61P 35/00; C07K 14/705; C07K 16/32; C12N 15/86; C12N 15/88

(52) U.S. Cl. CPC A61K 35/17; A61K 40/11; A61K 40/15; A61K 40/32; A61K 40/4243; A61P 35/00; C07K 14/70517; C07K 16/32; C12N 15/86; C12N 15/88; C07K 2317/565; C12N 2740/00043

(57) ABSTRACT

The presently disclosed subject matter provides novel T cell receptors (TCRs) that target an EWSR1/WT1 fusion protein. The presently disclosed subject matter further provides cells comprising such TCRs, and methods of using such cells for treating cancers associated with EWSR1/WT1 fusion protein.

### CROSS-REFERENCE TO RELATED APPLICATIONS

This application is a continuation application of International Patent Application No. PCT/US2023/078139, filed Oct. 30, 2023, which claims priority to U.S. Provisional Application No. 63/381,328, filed Oct. 28, 2022, the contents of each of which are incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document submitted herewith encoded as XML in UTF-8 text. The electronic document, created on Apr. 28, 2025, is entitled “0727341728.xml” and is 49,568 bytes in size.

### 1. INTRODUCTION

The presently disclosed subject matter provides novel T cell receptors (TCRs) that target EWSR1/WT1 fusion proteins. The presently disclosed subject matter further provides cells comprising such TCRs, and methods of using such cells for treating cancers associated with an EWSR1/WT1 fusion protein.

### 2. BACKGROUND OF THE INVENTION

Cell-based immunotherapy is a therapy with curative potential for the treatment of cancer. T cells and other immune cells can be modified to target tumor antigens through the introduction of genetic material coding for T cell receptors (TCRs) that confer specificity to selected antigens. Targeted T cell therapy using specific TCRs has shown clinical success in treating multiple solid and hematologic malignancies.

Sarcomas are a heterogenous group of malignancies of mesenchymal cell origin that are notoriously difficult to treat and often carry poor prognoses. Sarcomas typically harbor many fewer non-synonymous somatic mutations (NSSMs), or point mutations, compared with other solid cancers. Because a subset of NSSMs generate T cell activating neoantigens, this observation has been advanced as a biologic explanation for why sarcomas are thought to be immunologically “cold” with typically poor responses to immune checkpoint inhibitors (ICI). However, many sarcomas are characterized by a different type of gene alteration, termed oncogenic fusion, in which one gene is genetically fused to another. A prototypical fusion-driven sarcoma is desmoplastic small round cell tumor (DSRCT), which is characterized by a pathognomonic EWSR1-WT1 fusion event. Oncogenic fusion proteins might yield particularly immunogenic subset of neoantigens because they create novel peptide sequences that diverge significantly from self-proteins. Targeting fusion-derived neoantigens can therefore serve as an innovative new strategy to bring the curative potential of immunotherapy to sarcomas. Accordingly, there remains a critical unmet need for novel therapeutic strategies to identify and generate TCRs targeting oncogenic fusion proteins, and for strategies capable of inducing potent cancer eradication with minimal risk of normal tissue toxicity.

### 3. SUMMARY OF THE INVENTION

The presently disclosed subject matter provides a recombinant T cell receptor (TCR) that binds to an EWSR1/WT1 peptide. In certain embodiments, the EWSR1/WT1 peptide comprises a junctional amino acid sequence of the fusion protein between EWS and WT1. In certain embodiments, the EWSR1/WT1 peptide comprises or consists of the amino acid sequence set forth in SEQ ID NO: 3, SEQ ID NO: 4, SEQ ID NO: 5, SEQ ID NO: 6, SEQ ID NO: 7, or SEQ ID NO: 8.

In certain embodiments, the EWSR1/WT1 peptide is associated with an HLA class I complex. In certain embodiments, the HLA class I complex is selected from an HLA-A, an HLA-B, and an HLA-C. In certain embodiments, the HLA class I complex is an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*33, HLA-A*66, HLA-A*68, and HLA-A*74. In certain embodiments, the HLA-A*03 superfamily member is HLA-A*03. In certain embodiments, the HLA-A*03 molecule is selected from the group consisting of HLA-A*0301, HLA-A*0302, and HLA-A*0305. In certain embodiments, the HLA-A*03 superfamily member is HLA-A*11. In certain embodiments, the HLA-A*11 molecule is selected from the group consisting of HLA-A*1101, HLA-A*1102, HLA-A*1104, and HLA-A*1105.

In certain embodiments, the TCR comprises an extracellular domain that binds to the EWSR1/WT1 peptide, wherein the extracellular domain comprises an α chain and a β chain, wherein the α chain comprises an α chain variable region and a chain constant region, and the β chain comprises a β chain variable region and a β chain constant region.

In certain embodiments,
a) the α chain variable region comprises a CDR3 comprising the amino acid sequence set forth in SEQ ID NO: 9 or a conservative modification thereof, and the β chain variable region comprises a CDR3 comprising the amino acid sequence set forth in SEQ ID NO: 10 or a conservative modification thereof;
b) the α chain variable region comprises a CDR3 comprising the amino acid sequence set forth in SEQ ID NO: 13 or a conservative modification thereof, and the β chain variable region comprises a CDR3 comprising the amino acid sequence set forth in SEQ ID NO: 14 or a conservative modification thereof;
c) the α chain variable region comprises a CDR3 comprising the amino acid sequence set forth in SEQ ID NO: 17 or a conservative modification thereof, and the β chain variable region comprises a CDR3 comprising the amino acid sequence set forth in SEQ ID NO: 18 or a conservative modification thereof; or
d) the α chain variable region comprises a CDR3 comprising the amino acid sequence set forth in SEQ ID NO: 41 or a conservative modification thereof, and the β chain variable region comprises a CDR3 comprising the amino acid sequence set forth in SEQ ID NO: 44 or a conservative modification thereof.

In certain embodiments,
a) the α chain variable region comprises a CDR2 comprising the amino acid sequence set forth in SEQ ID NO: 30 or a conservative modification thereof, and the β chain variable region comprises a CDR2 comprising the amino acid sequence set forth in SEQ ID NO: 32 or a conservative modification thereof;
b) the α chain variable region comprises a CDR2 comprising the amino acid sequence set forth in SEQ ID NO: 34 or a conservative modification thereof, and the β chain variable region comprises a CDR2 comprising the amino acid sequence set forth in SEQ ID NO: 36 or a conservative modification thereof;
c) the α chain variable region comprises a CDR2 comprising the amino acid sequence set forth in SEQ ID NO: 38 or a conservative modification thereof, and the β chain variable region comprises a CDR2 comprising the amino acid sequence set forth in SEQ ID NO: 32 or a conservative modification thereof; or
d) the α chain variable region comprises a CDR2 comprising the amino acid sequence set forth in SEQ ID NO: 40 or a conservative modification thereof, and the β chain variable region comprises a CDR2 comprising the amino acid sequence set forth in SEQ ID NO: 43 or a conservative modification thereof.

In certain embodiments,
a) the α chain variable region comprises a CDR1 comprising the amino acid sequence set forth in SEQ ID NO: 29 or a conservative modification thereof, and the β chain variable region comprises a CDR1 comprising the amino acid sequence set forth in SEQ ID NO: 31 or a conservative modification thereof;
b) the α chain variable region comprises a CDR1 comprising the amino acid sequence set forth in SEQ ID NO: 33 or a conservative modification thereof, and the β chain variable region comprises a CDR1 comprising the amino acid sequence set forth in SEQ ID NO: 35 or a conservative modification thereof;
c) the α chain variable region comprises a CDR1 comprising the amino acid sequence set forth in SEQ ID NO: 37 or a conservative modification thereof, and the β chain variable region comprises a CDR1 comprising the amino acid sequence set forth in SEQ ID NO: 31 or a conservative modification thereof; or
d) the α chain variable region comprises a CDR1 comprising the amino acid sequence set forth in SEQ ID NO: 39 or a conservative modification thereof, and the β chain variable region comprises a CDR1 comprising the amino acid sequence set forth in SEQ ID NO: 42 or a conservative modification thereof.

In certain embodiments,
a) the α chain variable region comprises a CDR1 comprising the amino acid sequence set forth in SEQ ID NO: 29 or a conservative modification thereof, a CDR2 comprising the amino acid sequence set forth in SEQ ID NO: 30 or a conservative modification thereof, and a CDR3 comprising the amino acid sequence set forth in SEQ ID NO: 9 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 10 or a conservative modification thereof;
b) the α chain variable region comprises a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 13 or a conservative modification thereof; and the β chain variable region comprises a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 14 or a conservative modification thereof;
c) the α chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 17 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 18 or a conservative modification thereof;
d) the α chain variable region comprises a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof; and the β chain variable region comprises a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof.

In certain embodiments,
a) the α chain variable region a CDR1 comprising the amino acid sequence set forth in SEQ ID NO: 29, a CDR2 comprising the amino acid sequence set forth in SEQ ID NO: 30, and a CDR3 comprising the amino acid sequence set forth in SEQ ID NO: 9; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 10;
b) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 13; and the β chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 14;
c) the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18; or
d) the α chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41; and the β chain variable region comprises a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44.

In certain embodiments, the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18.

In certain embodiments,
a) the α chain variable region comprises a CDR1, a CDR2, and a CDR3 of the α chain variable region having the amino acid sequence set forth in SEQ ID NO: 11; and the β chain variable region comprises a CDR1, a CDR2, and a CDR3 of the β chain variable region having the amino acid sequence set forth in SEQ ID NO: 12;
b) the α chain variable region comprises a CDR1, a CDR2, and a CDR3 of the α chain variable region having the amino acid sequence set forth in SEQ ID NO: 15; and the β chain variable region comprises a CDR1, a CDR2, and a CDR3 of the β chain variable region having the amino acid sequence set forth in SEQ ID NO: 16;
c) the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20; or
d) the α chain variable region comprises a CDR1, a CDR2, and a CDR3 of the α chain variable region having the amino acid sequence set forth in SEQ ID NO: 45; and the β chain variable region comprises a CDR1, a CDR2, and a CDR3 of the β chain variable region having the amino acid sequence set forth in SEQ ID NO: 46.

In certain embodiments, the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20.

In certain embodiments, the α chain variable region comprises an amino acid sequence that is at least about 80% homologous or identical to the amino acid sequence set forth in SEQ ID NO: 11, SEQ ID NO: 15, SEQ ID NO: 19, or SEQ ID NO: 45. In certain embodiments, the α chain variable region comprises the amino acid sequence set forth in SEQ ID NO: 11, SEQ ID NO: 15, SEQ ID NO: 19, or SEQ ID NO: 45. In certain embodiments, the α chain variable region comprises the amino acid sequence set forth in SEQ ID NO: 19.

In certain embodiments, the β chain variable region comprises an amino acid sequence that is at least about 80% homologous or identical to the amino acid sequence set forth in SEQ ID NO: 12, SEQ ID NO: 16, SEQ ID NO: 20, or SEQ ID NO: 46. In certain embodiments, the β chain variable region comprises the amino acid sequence set forth in SEQ ID NO: 12, SEQ ID NO: 16, SEQ ID NO: 20, or SEQ ID NO: 46. In certain embodiments, the β chain variable region comprises the amino acid sequence set forth in SEQ ID NO: 20.

In certain embodiments,
a) the α chain variable region comprises an amino acid sequence that is at least about 80% homologous or identical to the amino acid sequence set forth in SEQ ID NO: 11, and the β chain variable region comprises an amino acid sequence that is at least about 80% homologous or identical to the amino acid sequence set forth in SEQ ID NO: 12;
b) the α chain variable region comprises an amino acid sequence that is at least about 80% homologous or identical to the amino acid sequence set forth in SEQ ID NO: 15, and the β chain variable region comprises an amino acid sequence that is at least about 80% homologous or identical to the amino acid sequence set forth in SEQ ID NO: 16;
c) the α chain variable region comprises an amino acid sequence that is at least about 80% homologous or identical to the amino acid sequence set forth in SEQ ID NO: 19, and the β chain variable region comprises an amino acid sequence that is at least about 80% homologous or identical to the amino acid sequence set forth in SEQ ID NO: 20;
d) the α chain variable region comprises an amino acid sequence that is at least about 80% homologous or identical to the amino acid sequence set forth in SEQ ID NO: 45, and the β chain variable region comprises an amino acid sequence that is at least about 80% homologous or identical to the amino acid sequence set forth in SEQ ID NO: 46.

In certain embodiments,
a) the α chain variable region comprises the amino acid sequence set forth in SEQ ID NO: 11, and the β chain variable region comprises the amino acid sequence set forth in SEQ ID NO: 12;
b) the α chain variable region comprises the amino acid sequence set forth in SEQ ID NO: 15, and the β chain variable region comprises the amino acid sequence set forth in SEQ ID NO: 16;
c) the α chain variable region comprises the amino acid sequence set forth in SEQ ID NO: 19, and the β chain variable region comprises the amino acid sequence set forth in SEQ ID NO: 20; or
d) the α chain variable region comprises the amino acid sequence set forth in SEQ ID NO: 45, and the β chain variable region comprises the amino acid sequence set forth in SEQ ID NO: 46.

In certain embodiments, the α chain variable region comprises the amino acid sequence set forth in SEQ ID NO: 19, and the β chain variable region comprises the amino acid sequence set forth in SEQ ID NO: 20.

In certain embodiments, the extracellular domain binds to the same EWSR1/WT1 peptide as a reference TCR or a functional fragment thereof, wherein the reference TCR or functional fragment thereof comprises an α chain variable region and a β chain variable region, wherein:
a) the α chain variable region comprises a CDR3 comprising the amino acid sequence set forth in SEQ ID NO: 9; and the β chain variable region comprises a CDR3 comprising the amino acid sequence set forth in SEQ ID NO: 10;
b) the α chain variable region comprises a CDR3 comprising the amino acid sequence set forth in SEQ ID NO: 13; and the β chain variable region comprises a CDR3 comprising the amino acid sequence set forth in SEQ ID NO: 14;
c) the α chain variable region comprises a CDR3 comprising the amino acid sequence set forth in SEQ ID NO: 17; and the β chain variable region comprises a CDR3 comprising the amino acid sequence set forth in SEQ ID NO: 18; or
d) the α chain variable region comprises the amino acid sequence set forth in SEQ ID NO: 45, and the β chain variable region comprises the amino acid sequence set forth in SEQ ID NO: 46.

In certain embodiments, the recombinant TCR is recombinantly expressed, and/or expressed from a vector.

In certain embodiments, the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 21 or SEQ ID NO: 22. In certain embodiments, the α chain constant region comprises the amino acid sequence set forth in SEQ ID NO: 21 or SEQ ID NO: 22.

In certain embodiments, the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23, SEQ ID NO: 24, SEQ ID NO: 25, or SEQ ID NO: 26. In certain embodiments, the β chain constant region comprises the amino acid sequence set forth in SEQ ID NO: 23, SEQ ID NO: 24, SEQ ID NO: 25, or SEQ ID NO: 26.

The presently disclosed subject matter also provides nucleic acids encoding the T cell receptor (TCR) disclosed herein. Further, the presently disclosed subject matter provides cells comprising the TCR or the nucleic acid disclosed herein. In certain embodiments, the cell is transduced with the TCR. In certain embodiments, the TCR is constitutively expressed on the surface of the cell.

In certain embodiments, the cell is an immunoresponsive cell. In certain embodiments, the cell is selected from the group consisting of a T cell, a Natural Killer (NK) cell, and a pluripotent stem cell from which a lymphoid cell may be differentiated. In certain embodiments, the cell is a T cell. In certain embodiments, the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell. In certain embodiments, the T cell is a γδ T cell. In certain embodiments, the T cell is a NK-T cell. In certain embodiments, the cell is an NK cell.

In certain embodiments, the TCR is encoded by a nucleic acid integrated at a locus within the genome of the cell. In certain embodiments, the nucleic acid is integrated at a locus within the genome of the cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is selected from a PDCD1 locus, a CBLB locus, a CISH locus, and a RASA2 locus. In certain embodiments, the locus is a genomic safe harbor. In certain embodiments, the cell further comprises a recombinant or exogenous co-receptor. In certain embodiments, the co-receptor is a CD8 co-receptor. In certain embodiments, the CD8 co-receptor comprises an α chain and a β chain. In certain embodiments, the α chain comprises the amino acid sequence set forth in SEQ ID NO: 48. In certain embodiments, the β chain comprises the amino acid sequence set forth in SEQ ID NO: 49. In certain embodiments, the co-receptor is a CD4 co-receptor.

Moreover, the presently disclosed subject matter provides compositions comprising the cells disclosed herein. In certain embodiments, the composition is a pharmaceutical composition further comprising a pharmaceutically acceptable carrier.

The presently disclosed subject matter provides a vector or a lipid nanoparticle comprising the nucleic acids disclosed herein. In certain embodiments, the vector is a γ-retroviral vector.

The presently disclosed subject matter further provides a method for producing a cell that binds to an EWSR1/WT1 peptide, comprising introducing into the cell the nucleic acids, the vectors, or the lipid nanoparticles disclosed herein.

The presently disclosed subject matter provides methods of treating and/or preventing a tumor associated with EWSR1/WT1 fusion protein in a subject, comprising administering to the subject an effective amount of the cell, the composition, the vector, or the lipid nanoparticle disclosed herein. Additionally or alternatively, the presently disclosed subject matter provides methods of reducing tumor burden in a subject having a tumor associated with EWSR1/WT1 fusion protein, comprising administering to the subject an effective amount of the cell, the composition, the vector, or the lipid nanoparticle disclosed herein. In certain embodiments, the EWSR1/WT1 peptide comprises a junctional amino acid sequence of the fusion protein between EWS and WT1.

In certain embodiments, the tumor is selected from the group consisting of desmoplastic small round cell tumor (DSRCT), soft tissue sarcoma, colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

In certain embodiments, the tumor is desmoplastic small round cell tumor (DSRCT).

In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*33, HLA-A*66, HLA-A*68, and HLA-A*74. In certain embodiments, the HLA-A*03 superfamily member is HLA-A*03. In certain embodiments, the HLA-A*03 molecule is selected from the group consisting of HLA-A*0301, HLA-A*0302, and HLA-A*0305. In certain embodiments, the HLA-A*03 superfamily member is HLA-A*11. In certain embodiments, the HLA-A*11 molecule is selected from the group consisting of HLA-A*1101, HLA-A*1102, HLA-A*1104, and HLA-A*1105. In certain embodiments, the subject is a human.

In certain non-limiting embodiments, the presently disclosed subject matter provide the cell, the composition, the vector, or the lipid nanoparticle disclosed herein for use in treating and/or preventing a tumor associated with EWSR1/WT1 fusion protein in a subject. Additionally or alternatively, the cell, the composition, the vector, or the lipid nanoparticle disclosed herein are for use in reducing tumor burden in a subject having a tumor associated with EWSR1/WT1 fusion protein. In certain embodiments, the EWSR1/WT1 peptide comprises a junctional amino acid sequence of the fusion protein between EWS and WT1. In certain embodiments, the tumor is selected from the group consisting of desmoplastic small round cell tumor (DSRCT), soft tissue sarcoma, colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma. In certain embodiments, the tumor is desmoplastic small round cell tumor (DSRCT). In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*33, HLA-A*66, HLA-A*68, and HLA-A*74. In certain embodiments, the HLA-A*03 superfamily member is HLA-A*03. In certain embodiments, the HLA-A*03 molecule is selected from the group consisting of HLA-A*0301, HLA-A*0302, and HLA-A*0305. In certain embodiments, the HLA-A*03 superfamily member is HLA-A*11. In certain embodiments, the HLA-A*11 molecule is selected from the group consisting of HLA-A*1101, HLA-A*1102, HLA-A*1104, and HLA-A*1105. In certain embodiments, the subject is a human.

### 4. BRIEF DESCRIPTION OF THE FIGURES

The following Detailed Description, given by way of example but not intended to limit the invention to specific embodiments described, may be understood in conjunction with the accompanying drawings.

FIG. 1 illustrates the strategy used to clone the candidate NeoAg TCRs into retroviral vectors for transduction into T cells.

FIGS. 2A and 2B illustrate the antigen and HLA specificity of the three candidate TCRs (TCR 12, TCR 14, and TCR 15) to the EWSR1/WT1 fusion derived peptide (Fusion) in the context of the HLA-A03 allele. FIG. 2A shows that TCR12 leads to an increase in relative IFNG expression induced by EWSR1/WT1 fusion derived peptide in the context of HLA-A*03 in comparison to controls (no Ag or Control (WT1)). FIG. 2B illustrates that TCR14 and TCR15 specifically bind to the EWSR1/WT1 fusion derived peptide (Fusion) in the context of HLA-A*03 (Fusion+A*03), but not in EWSR1/WT1⁻/HLA-mismatched target cells (Fusion+A*02).

FIG. 3 illustrates functional validation for all tested TCRs (TCR12, TCR14, and TCR15) by measuring the dose response curve of Max TNFα as a function of EWSR1/WT1 peptide concentration.

FIG. 4 illustrates that TCR 15 recognizes the EWSR1/WT1 derived NeoAg when presented by either HLA-A03 or HLA-A11 alleles.

FIG. 5 illustrates Ala/Gly peptide scanning mutagenesis data demonstrating the specificity of each candidate TCR (TCR12, TCR14, and TCR15) to the EWSR1/WT1 fusion peptide.

FIGS. 6A-6C illustrate antigen and HLA specificity of the TCRs disclosed herein. FIG. 6A shows dual dextramer staining of polyclonal T cells transduced with candidate TCRs specifically bind fusion-NeoAg loaded dextramers but not viral peptide-loaded dextramers. Cells are gated on live CD3⁺, CD8⁺ transduced lymphocytes. FIG. 6B shows polyclonal CD8⁺ T cells transduced with candidate TCRs were co-cultured with APCs expressing 1) the requisite HLA allele and the EWSR1-WT1 fusion, 2) requisite HLA allele and unfused WT1, or 3) mismatched HLA allele and the EWSR1-WT1 fusion. Resulting TNFα expression was measured by intracellular flow cytometry. Cells are gated on live, CD3+, CD8+, transduced lymphocytes. FIG. 6C shows polyclonal CD8⁺ T cells transduced with candidate TCRs are co-cultured with APCs pulsed with varying concentrations of EWSR1-WT1 fusion NeoAg peptide. Subsequent TNFα production is measured by intracellular flow cytometry and IC50 calculated by non-linear fit of resulting titration curve. Data are presented as % max TNFα production (defined as TNFα production with 100 μM peptide pulse). SEQ ID NO: 4, SEQ ID NO: 47, and SEQ ID NO: 51 are depicted in FIG. 6A.

FIG. 7 illustrates X-scan of NeoAg peptide set forth in SEQ ID NO: 4 to evaluate for specificity for sequence recognition by candidate TCRs. Peptide substitution library in which each position of neoepitope was systematically and individually substituted with every other possible amino acid at that position (listed on Y axis) was generated. APCs expressing requisite HLA allele were pulsed with each peptide separately and cocultured with candidate TCRs. Culture supernatant was collected and IFNγ production was measured by ELISA. Heatmaps reflect median values among biologic triplicates except for TCR15 which depicts single representative values. IFNγ production is normalized to IFNγ produced by native residue at each position (i.e., fusion peptide). Native residue for each position is labeled on the X axis and indicated by a red box in the table. Ratios greater than 1.5 (indicating more than 1.5×IFNγ produced by native/cognate residue) are in green.

FIGS. 8A and 8B illustrate polyclonal CD8⁺ T cells transduced with candidate TCRs were cultured with DSRCT cells expressing GFP (E:T ratio of 1:2). GFP Area normalized to T0 was used as a measure of DSRCT cell growth (>1) or lysis (<1). HLA⁺ DSRCT cells were lysed by candidate TCRs (FIG. 8A). DSRCT lysis was attenuated in the presence of Class I blocking antibody or HLA⁻ DSRCT cells. Viral protein specific TCRs with the same HLA restriction did not lyse HLA⁺ DSRCT cells (FIG. 8B).

FIGS. 9A-9D illustrate antigen and HLA specificity of the TCRs disclosed herein. FIG. 9A shows polyclonal CD8⁺ T cells transduced with candidate TCR15 are co-cultured with either HLA-A*03 or HLA-A*11 expressing APCs pulsed with varying concentrations of EWSR1-WT1 fusion NeoAg peptide. Subsequent TNFα production is measured by intracellular flow cytometry and data is presented as % max TNFα production (defined as TNFα production with 100 μM peptide pulse). FIG. 9B shows polyclonal CD4⁺ T cells were transduced with TCR15 (blue) and a bicistronic construct containing TCR15 and CD8αβ (red) allowing for exogenous CD8 expression. CD8+ T cells with TCR15 or TCR15+CD8αβ were co-cultured with APCs expressing either HLA-A*03 or HLA-A*11 and pulsed with the fusion NeoAg. Resulting TNFα expression was measured by intracellular flow cytometry. Cells are gated on live, CD3⁺, CD8⁺ transduced, lymphocytes. FIG. 9C shows polyclonal CD8+ T cells transduced with candidate TCRs were cultured with DSRCT cells expressing GFP (E:T ratio of 1:2). GFP Area normalized to T0 was used as a measure of DSRCT cell growth (>1) or lysis (<1). FIG. 9D shows polyclonal CD8+ T cells transduced with candidate TCRs with (triangle) and without (hexagon for TCR15 or diamond for TCR 16) CD8αβ were cultured with DSRCT cells expressing GFP (E:T ratio of 1:2). GFP Area normalized to T0 was used as a measure of DSRCT cell growth (>1) or lysis (<1).

FIG. 10 depicts a summary of the TCRs disclosed herein. SEQ ID Nos: 9, 10, 13, 14, 17, 18, 41, and 44 are depicted as well.

### 5. DETAILED DESCRIPTION OF THE INVENTION

The presently disclosed subject matter provides T cell receptors (TCRs) targeting EWSR1/WT1 fusion proteins. Furthermore, the presently disclosed subject matter provides cells (e.g., T cells) comprising the EWSR1/WT1-targeted TCRs, and methods of using such cells for treating tumors associated with EWSR1/WT1 fusion protein(s).

For purposes of clarity of disclosure and not by way of limitation, the detailed description is divided into the following subsections:
5.1. Definitions;
5.2. EWSR1/WT1;
5.3. TCRs;
5.4. Cells;
5.5. Nucleic Acids and Genetic Modifications of Cell;
5.6 Formulations and Administration;
5.7. Methods of Treatments;
5.8 Diagnostic and Prognostic Methods;
5.9. Kits; and
5.10. Exemplary Embodiments.

### 5.1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of a TCR, which are the hypervariable regions of TCR α-chain and β-chain. Generally, a TCR comprises three CDRs in the α-chain variable region and three CDRs in the β-chain variable region. CDRs provide the majority of contact residues for the binding of the TCR to the antigen or epitope. CDRs regions can be delineated using the Kabat system (Kabat, E. A., et al. (1991) Sequences of Proteins of Immunological Interest, Fifth Edition, U.S. Department of Health and Human Services, NIH Publication No. 91-3242), the Chothia numbering system (Chothia et al., J Mol Biol. (1987) 196:901-17), the AbM numbering system (Abhinandan et al., Mol. Immunol. 2008, 45, 3832-3839), or the IMGT numbering system (accessible at http://www.imgt.org/IMGTScientificChart/Numbering/IMGTIGVLsuperfamily.html #table1, http://www.imgt.org/IMGTScientificChart/Nomenclature/IMGT-FRCDRdefinition.html). In certain embodiments, the CDRs regions are delineated using the IMGT numbering system.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally, or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, word length=3 to obtain amino acid sequences homologous to the specified sequences disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed TCR comprising the amino acid sequence. Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered TCR can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the term “disease” refers to any condition or disorder that damages or interferes with the normal function of a cell, tissue, or organ. Examples of diseases include neoplasm or pathogen infection of a cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a tumor), prevent or delay the recurrence of a tumor, or otherwise reduce the pathological consequences of the disease (e.g., a tumor). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “tumor” refers to an abnormal mass of tissue that forms when cells grow and divide more than they should or do not die when they should. Tumors include benign tumors and malignant tumors (known as “cancers”). Benign tumors may grow large but do not spread into, or invade, nearby tissues or other parts of the body. Malignant tumors can spread into, or invade, nearby tissues. They can also spread to other parts of the body through the blood and lymph systems. Tumor is also called neoplasm. In certain embodiments, the tumor is cancer.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “isolated cell” refers to a cell that is separated from the molecular and/or cellular components that naturally accompany the cell.

As used herein, the term “antigen” refers to any substance that the body (e.g., human body) regards as foreign and that therefore elicits an immune response (e.g., formation of specific antibodies capable of binding to it).

As used herein, the term “neoantigen” or “NeoAg” refers to aberrant antigens, which can be recognized by immune cells and result from mutations in cancer cell genes (e.g., caused by genetic instability during carcinogenesis). Neoantigens can arise through diverse mechanisms including, but not limited to, gene fusions, missense mutations, alternative splicing and indels. Neoantigens can be presented on the cell surface and subsequently recognized by T cells under the action of major histocompatibility complex (MHC) molecules.

As used herein, the term “treating” or “treatment” refers to clinical intervention in an attempt to alter the disease course of the individual or cell being treated and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses; and non-human primates such as apes and monkeys.

A “recombinant TCR” refers to a TCR that binds to an antigen (e.g., EWSR1/WT1) and that is prepared, expressed, generated, and/or isolated using recombinant technologies. For example, but without any limitation, a recombinant TCR is expressed in a cell (e.g., a T cell, a NK cell) including an exogenous recombinant expression vector (e.g., plasmid, nanoplasmid, retroviral vector, etc.). Additional information regarding methods and procedures to prepare, express, generate, and/or isolate recombinant TCRs can be found in Edes al., “TCR and CAR engineering of primary human T cells.” Gene Therapy of Cancer: Methods and Protocols. New York, NY: Springer US, 2022. 85-93, which is incorporated by reference in its entirety.

As used herein, the term “binds to,” when referring to a T cell receptor (TCR), means that the TCR binds to a desired antigen (e.g., a EWSR1/WT1 exon 7/8 fusion peptide) presented by the human leukocyte antigen (HLA) complex either in multimer form (e.g., dextramer) or at the surface of cells (e.g., antigen-presenting cells or tumor cells).

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

### 5.2. EWSR1/WT1

EWS is a protein that in humans is encoded by the EWSR1 gene on human chromosome 22, specifically 22q12.2. It was demonstrated that one of the EWS transcripts can bind to RNA in vitro, and specifically to poly-G and poly-U (Ohno et al., Oncogene 9, no. 10 (1994): 3087-3097). The RNA-binding activity was localized to the C-terminal 86 amino acids that constitute an RGG box. Thus, the N-terminal domain of EWS, which is involved in chromosome translocation, can regulate the specificity of RNA-binding activity of EWS. By mutation analysis of the EWS/ERG chimeric protein, it was found that the N-terminal EWS functions as a regulatory domain for the transcriptional activation properties of the chimeric protein (Ohno et al., Oncogene 9, no. 10 (1994): 3087-3097). In certain embodiments, the EWS protein comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 1. In certain embodiments, the EWS protein comprises or consists of the amino acid sequence set forth in SEQ ID NO: 1. SEQ ID NO: 1 is provided below.

[SEQ ID NO: 1]
MASTDYSTYSQAAAQQGYSAYTAQPTQGYAQTTQAYGQQSYGTYGQPTDV
SYTQAQTTATYGQTAYATSYGQPPTGYTTPTAPQAYSQPVQGYGTGAYDT
TTATVTTTQASYAAQSAYGTQPAYPAYGQQPAATAPTRPQDGNKPTETSQ
PQSSTGGYNQPSLGYGQSNYSYPQVPGSYPMQPVTAPPSYPPTSYSSTQP
TSYDQSSYSQQNTYGQPSSYGQQSSYGQQSSYGQQPPTSYPPQTGSYSQA
PSQYSQQSSSYGQQSSFRQDHPSSMGVYGQESGGFSGPGENRSMSGPDNR
GRGRGGFDRGGMSRGGRGGGRGGMGSAGERGGFNKPGGPMDEGPDLDLGP
PVDPDEDSDNSAIYVQGLNDSVTLDDLADFFKQCGVVKMNKRTGQPMIHI
YLDKETGKPKGDATVSYEDPPTAKAAVEWEDGKDFQGSKLKVSLARKKPP
MNSMRGGLPPREGRGMPPPLRGGPGGPGGPGGPMGRMGGRGGDRGGFPPR
GPRGSRGNPSGGGNVQHRAGDWQCPNPGCGNQNFAWRTECNQCKAPKPEG
FLPPPFPPPGGDRGRGGPGGMRGGRGGLMDRGGPGGMFRGGRGGDRGGFR
GGRGMDRGGFGGGRRGGPGGPPGPLMEQMGGRRGGRGGPGKMDKGEHRQE
RRDRPY

WT1 is a transcription factor that contains four zinc-finger motifs at the C-terminus and a proline/glutamine-rich DNA-binding domain at the N-terminus. It has an essential role in the normal development of the urogenital system, and it is mutated in a small subset of patients with Wilms tumor. This gene exhibits complex tissue-specific and polymorphic imprinting pattern, with biallelic, and monoallelic expression from the maternal and paternal alleles in different tissues (Yang et al., Leukemia 21, no. 5 (2007): 868-876). In certain embodiments, the WT1 protein comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 2. In certain embodiments, the WT1 protein comprises or consists of the amino acid sequence set forth in SEQ ID NO: 2. SEQ ID NO: 2 is provided below.

[SEQ ID NO: 2]
MGSDVRDLNALLPAVPSLGGGGGCALPVSGAAQWAPVLDFAPPGASAYGS
LGGPAPPPAPPPPPPPPPHSFIKQEPSWGGAEPHEEQCLSAFTVHFSGQF
TGTAGACRYGPFGPPPPSQASSGQARMEPNAPYLPSCLESQPAIRNQGYS
TVTFDGTPSYGHTPSHHAAQFPNHSFKHEDPMGQQGSLGEQQYSVPPPVY
GCHTPTDSCTGSQALLLRTPYSSDNLYQMTSQLECMTWNQMNLGATLKGV
AAGSSSSVKWTEGQSNHSTGYESDNHTTPILCGAQYRIHTHGVERGIQDV
RRVPGVAPTLVRSASETSEKRPFMCAYPGCNKRYFKLSHLQMHSRKHTGE
KPYQCDEKDCERRESRSDQLKRHQRRHTGVKPFQCKTCQRKFSRSDHLKT
HTRTHTGKTSEKPFSCRWPSCQKKFARSDELVRHHNMHQRNMTKLQLAL

In many tumors, a rearrangement of EWSR1 and WT1, which creates a fusion protein, has been observed. EWSR1/WT1 (“EWSR1/WT1”) is an oncogenic fusion protein found in almost all cases of desmoplastic small round cell tumor (DSRCT) and in other tumors which contain a characteristic t(11;22) (p13;q12) chromosomal translocation involving the EWSR1 and WT1 genes. This translocation results in the fusion of the N-terminus of the EWSR1 protein with the C-terminus of the WT1 protein and drives tumorigenesis. The breakpoint between the proteins creates a novel fusion protein that diverges significantly from self-proteins and is unique to the tumor. Additional information regarding the EWSR1/WT1 fusion protein and its rearrangements can be found in Worley et al., Cancer research 61, no. 18 (2001): 6868-6875, the content of which is incorporated by reference in its entirety. In certain embodiments, the EWSR1/WT1 protein comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 28. In certain embodiments, the EWSR1/WT1protein comprises or consists of the amino acid sequence set forth in SEQ ID NO: 28. SEQ ID NO: 28 is provided below.

[SEQ ID NO: 28]
MASTDYSTYSQAAAQQGYSAYTAQPTQGYAQTTQAYGQQSYGTYGQPTDV
SYTQAQTTATYGQTAYATSYGQPPTGYTTPTAPQAYSQPVQGYGTGAYDT
TTATVTTTQASYAAQSAYGTQPAYPAYGQQPAATAPTRPQDGNKPTETSQ
PQSSTGGYNQPSLGYGQSNYSYPQVPGSYPMQPVTAPPSYPPTSYSSTQP
TSYDQSSYSQQNTYGQPSSYGQQSSYGQQSSYGQQPPTSYPPQTGSYSQA
PSQYSQQSSSYGQQSEKPYQCDFKDCERRFSRSDQLKRHQRRHTGVKPFQ
CKTCQRKFSRSDHLKTHTRTHTGKTSEKPFSCRWPSCQKKFARSDELVRH
HNMHQRNMTKLQLAL

### 5.3. T-Cell Receptor (TCR)

A TCR is a disulfide-linked heterodimeric protein consisting of two variable chains expressed as part of a non-covalent complex with the invariant CD3 chain molecules (CD3δ, CD3ε, CD3γ, CD3ζ). A TCR is found on the surface of T cells and is responsible for recognizing antigens bound to major histocompatibility complex (MHC) molecules. In certain embodiments, a TCR comprises an α chain and a β chain (encoded by TRA and TRB, respectively). In certain embodiments, a TCR comprises a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its cognate peptide antigen/MHC (peptide/MHC), the T cell expressing the TCR complex is activated.

The presently disclosed subject matter provides recombinant TCRs. In certain embodiments, the recombinant TCR differs from any naturally occurring TCR by at least one amino acid residue. In certain embodiments, the recombinant TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the recombinant TCR is modified from a naturally occurring TCR by at least one amino acid residue. In certain embodiments, the recombinant TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the presently disclosed TCR targets or binds to a EWSR1/WT1 peptide comprising the junctional amino acid sequence of the fusion protein between EWS and WT1. In certain embodiments, the EWSR1/WT1 peptide comprises or consists of the amino acid sequence set forth in SEQ ID NO: 3. In certain embodiments, the EWSR1/WT1 peptide comprises or consists of the amino acid sequence set forth in SEQ ID NO: 4. In certain embodiments, the EWSR1/WT1 peptide comprises or consists of the amino acid sequence set forth in SEQ ID NO: 5. In certain embodiments, the EWSR1/WT1 peptide comprises or consists of the amino acid sequence set forth in SEQ ID NO: 6. In certain embodiments, the EWSR1/WT1 peptide comprises or consists of the amino acid sequence set forth in SEQ ID NO: 7. In certain embodiments, the EWSR1/WT1 peptide comprises or consists of the amino acid sequence set forth in SEQ ID NO: 8. In certain embodiments, the presently disclosed TCR does not bind to a wildtype EWS. In certain embodiments, the presently disclosed TCR does not bind to a wildtype WT1. SEQ ID NOs: 3-8 are provided below.

[SEQ ID NO: 3]
SSSYGQQSEKPYQCDFK
[SEQ ID NO: 4]
SSYGQQSEK
[SEQ ID NO: 5]
SSSYGQQSEK
[SEQ ID NO: 6]
YGQQSEKPY
[SEQ ID NO: 7]
SEKPYQCDF
[SEQ ID NO: 8]
SYGQQSEKPY

In certain embodiments, the presently disclosed TCR targets or binds to a EWSR1/WT1 peptide comprising the junctional amino acid sequence of the fusion protein and associated with an HLA-A*03 superfamily (e.g., in an HLA-A*01 superfamily dependent manner). In certain embodiments, the HLA-A*03 superfamily member is selected from the group consisting of HLA-A*03, HLA-A*11, HLA-A*33, HLA-A*66, HLA-A*68, and HLA-A*74. In certain embodiments, the presently disclosed TCR targets or binds to a EWSR1/WT1 peptide associated with an HLA-A*03 molecule. In certain embodiments, the HLA-A*03 molecule is selected from the group consisting of HLA-A*0301, HLA-A*0302, and HLA-A*0305.

In certain embodiments, the presently disclosed TCR targets or binds to a EWSR1/WT1 peptide comprising the junctional amino acid sequence of the fusion protein and associated with an HLA-A*11 molecule. In certain embodiments, the HLA-A*11 molecule is selected from the group consisting of HLA-A*1101, HLA-A*1102, HLA-A*1104, and HLA-A*1105.

In certain embodiments, the presently disclosed TCR is a recombinant TCR.

### 5.3.1. TCRs

### 5.3.1.1. Variable Regions

In certain embodiments, the extracellular domain of the TCR comprises an α chain variable region comprising a CDR1, a CDR2, and a CDR3. In certain embodiments, the extracellular domain of the TCR comprises an α chain variable region comprising a CDR1 comprising the amino acid sequence set forth in SEQ ID NO: 29 or a conservative modification thereof, a CDR2 comprising the amino acid sequence set forth in SEQ ID NO: 30 or a conservative modification thereof, and a CDR3 comprising the amino acid sequence set forth in SEQ ID NO: 9 or a conservative modification thereof. SEQ ID NOs: 9, 29, and 30 are disclosed in Table 1. In certain embodiments, the α chain variable region comprises a CDR1 comprising the amino acid sequence set forth in SEQ ID NO: 29, a CDR2 comprising the amino acid sequence set forth in SEQ ID NO: 30, and a CDR3 comprising the amino acid sequence set forth in SEQ ID NO: 9.

In certain embodiments, the extracellular domain of the TCR comprises an β chain variable region comprising a CDR1, a CDR2, and a CDR3. In certain embodiments, the extracellular domain of the TCR comprises an β chain variable region comprising a CDR1 comprising the amino acid sequence set forth in SEQ ID NO: 31 or a conservative modification thereof a CDR3 comprising the amino acid sequence set forth in SEQ ID NO: 32 or a conservative modification thereof a CDR3 comprising the amino acid sequence set forth in SEQ ID NO: 10 or a conservative modification thereof. SEQ ID NOs: 10, 31 and 32 are disclosed in Table 1. In certain embodiments, the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 10.

In certain embodiments, the α chain variable region comprises a CDR1, a CDR2, and a CDR3; and the β chain variable region comprises a CDR1, a CDR2, and a CDR3. In certain embodiments, the α chain variable region comprises a CDR1 comprising the amino acid sequence set forth in SEQ ID NO: 29 or a conservative modification thereof, a CDR2 comprising the amino acid sequence set forth in SEQ ID NO: 30 or a conservative modification thereof, and a CDR3 comprising the amino acid sequence set forth in SEQ ID NO: 9 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 10 or a conservative modification thereof. In certain embodiments, the α chain variable region comprises a CDR1 comprising the amino acid sequence set forth in SEQ ID NO: 29, a CDR2 comprising the amino acid sequence set forth in SEQ ID NO: 30, and a CDR3 comprising the amino acid sequence set forth in SEQ ID NO: 9; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 10.

In certain embodiments, the α chain variable region comprises a CDR1, a CDR2, and a CDR3 of the α chain variable region having the amino acid sequence set forth in SEQ ID NO: 11. In certain embodiments, the β chain variable region comprises a CDR1, a CDR2, and a CDR3 of the β chain variable region having the amino acid sequence set forth in SEQ ID NO: 12. In certain embodiments, the α chain variable region comprises a CDR1, a CDR2, and a CDR3 of the α chain variable region having the amino acid sequence set forth in SEQ ID NO: 11; and the β chain variable region comprises a CDR1, a CDR2, and a CDR3 of the β chain variable region having the amino acid sequence set forth in SEQ ID NO: 12.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 11. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 11. In certain embodiments, the α chain variable region comprises the amino acid sequence set forth in SEQ ID NO: 11. SEQ ID NO: 11 is provided in Table 1.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 12. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 12. In certain embodiments, the β chain variable region comprises the amino acid sequence set forth in SEQ ID NO: 12. SEQ ID NO: 12 is provided in Table 1.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 11; and the β chain variable region comprises an amino acid sequence that is at least about 80% (e.g., at least about 85%, at least about 90%, or at least about 95%) homologous or identical to the amino acid sequence set forth in SEQ ID NO: 12. In certain embodiments, the α chain variable region comprises the amino acid sequence set forth in SEQ ID NO: 11; and the β chain variable region comprises the amino acid sequence set forth in SEQ ID NO: 12.

In certain embodiments, the TCR is designated as “TCR12”. In certain embodiments, the TCR12 binds to a EWSR1/WT1 peptide comprising or consisting of the amino acid sequence set forth in SEQ ID NO: 4, SEQ ID NO: 5, SEQ ID NO: 6, SEQ ID NO: 7, or SEQ ID NO: 8.

In certain embodiments, the CDRs sequences described above including Table 1 are delineated using the IMGT numbering system.

TABLE 1
(TCR12)

| | |
|---|---|
| CDR1 α-chain | TRDTTYY |
| | [SEQ ID NO: 29] |
| CDR2 α-chain | RNSFDEQN |
| | [SEQ ID NO: 30] |
| CDR3 α-chain | CALSEMQGGSEKLVF |
| | [SEQ ID NO: 9] |
| CDR1 ß-chain | SGDLS |
| | [SEQ ID NO: 31] |
| CDR2 ß-chain | YYNGEE |
| | [SEQ ID NO: 32] |
| CDR3 ß-chain | CASSTSSGSPDTQYF |
| | [SEQ ID NO: 10] |
| α-chain | MLTASLLRAVIASICVVSSMAQKVTQAQTEISVVEK |
| variable | EDVTLDCVYETRDTTYYLFWYKQPPSGELVFLIRRN |
| | SFDEQNEISGRYSWNFQKSTSSFNFTITASQVVDSA |
| | VYFCALSEMQGGSEKLVFGKGTKLTVNP |
| | [SEQ ID NO: 11] |
| ß-chain | MGFRLLCCVAFCLLGAGPVDSGVTQTPKHLITATGQ |
| variable | RVTLRCSPRSGDLSVYWYQQSLDQGLQFLIHYYNGE |
| | ERAKGNILERFSAQQFPDLHSELNLSSLELGDSALY |
| | FCASSTSSGSPDTQYFGPGTRLTVL |
| | [SEQ ID NO: 12] |

In certain embodiments, the extracellular domain of the TCR comprises an α chain variable region comprising a CDR1, a CDR2, and a CDR3. In certain embodiments, the extracellular domain of the TCR comprises an α chain variable region comprising a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 13 or a conservative modification thereof. SEQ ID NOs: 13, 33, and 34 are disclosed in Table 2. In certain embodiments, the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 13.

In certain embodiments, the extracellular domain of the TCR comprises an β chain variable region comprising a CDR1, a CDR2, and a CDR3. In certain embodiments, the extracellular domain of the TCR comprises an β chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 14 or a conservative modification thereof. SEQ ID NOs: 14, 35, and 36 are disclosed in Table 2. In certain embodiments, the β chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 14.

In certain embodiments, the α chain variable region comprises a CDR1, a CDR2, and a CDR3; and the β chain variable region comprises a CDR1, a CDR2, and a CDR3. In certain embodiments, the α chain variable region comprises a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 13 or a conservative modification thereof; and the β chain variable region comprises a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 14 or a conservative modification thereof. In certain embodiments, the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 13; and the β chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 14.

In certain embodiments, the α chain variable region comprises a CDR1, a CDR2, and a CDR3 of the α chain variable region having the amino acid sequence set forth in SEQ ID NO: 15. In certain embodiments, the β chain variable region comprises a CDR1, a CDR2, and a CDR3 of the β chain variable region having the amino acid sequence set forth in SEQ ID NO: 16. In certain embodiments, the α chain variable region comprises a CDR1, a CDR2, and a CDR3 of the α chain variable region having the amino acid sequence set forth in SEQ ID NO: 15; and the β chain variable region comprises a CDR1, a CDR2, and a CDR3 of the β chain variable region having the amino acid sequence set forth in SEQ ID NO: 16.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 15. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 15. In certain embodiments, the α chain variable region comprises the amino acid sequence set forth in SEQ ID NO: 15. SEQ ID NO: 15 is provided in Table 2.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 16. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 16. In certain embodiments, the β chain variable region comprises the amino acid sequence set forth in SEQ ID NO: 16. SEQ ID NO: 16 is provided in Table 2.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 15; and the β chain variable region comprises an amino acid sequence that is at least about 80% (e.g., at least about 85%, at least about 90%, or at least about 95%) homologous or identical to the amino acid sequence set forth in SEQ ID NO: 16. In certain embodiments, the α chain variable region comprises the amino acid sequence set forth in SEQ ID NO: 15; and the β chain variable region comprises the amino acid sequence set forth in SEQ ID NO: 16.

In certain embodiments, the TCR is designated as “TCR14”. In certain embodiments, the TCR14 binds to a EWSR1/WT1 peptide comprising or consisting of the amino acid sequence set forth in SEQ ID NO: 4, SEQ ID NO: 5, SEQ ID NO: 6, SEQ ID NO: 7, or SEQ ID NO: 8.

In certain embodiments, the CDRs sequences described above including Table 2 are delineated using the IMGT numbering system.

TABLE 2
(TCR14)

| | |
|---|---|
| CDR1 α-chain | SIFNT |
| | [SEQ ID NO: 33] |
| CDR2 α-chain | LYKAGEL |
| | [SEQ ID NO: 34] |
| CDR3 α-chain | CAGQGASGTYKYIF |
| | [SEQ ID NO: 13] |
| CDR1 ß-chain | SGHNS |
| | [SEQ ID NO: 35] |
| CDR2 ß-chain | FNNNVP |
| | [SEQ ID NO: 36] |
| CDR3 ß-chain | CASSPGTSDEFF |
| | [SEQ ID NO: 14] |
| α-chain | MLLEHLLIILWMQLTWVSGQQLNQSPQSMFIQEGED |
| variable | VSMNCTSSSIFNTWLWYKQDPGEGPVLLIALYKAGE |
| | LTSNGRLTAQFGITRKDSFLNISASIPSDVGIYFCA |
| | GQGASGTYKYIFGTGTRLKVLA |
| | [SEQ ID NO: 15] |
| ß-chain | MDSWTFCCVSLCILVAKHTDAGVIQSPRHEVTEMGQ |
| variable | EVTLRCKPISGHNSLFWYRQTMMRGLELLIYFNNNV |
| | PIDDSGMPEDRFSAKMPNASFSTLKIQPSEPRDSAV |
| | YFCASSPGTSDEFFGPGTRLTVL |
| | [SEQ ID NO: 16] |

In certain embodiments, the extracellular domain of the TCR comprises an α chain variable region comprising a CDR1, a CDR2, and a CDR3. In certain embodiments, the extracellular domain of the TCR comprises an α chain variable region comprising a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 17 or a conservative modification thereof. SEQ ID NOs: 17, 37, and 38 are disclosed in Table 3. In certain embodiments, the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17.

In certain embodiments, the extracellular domain of the TCR comprises an β chain variable region comprising a CDR1, a CDR2, and a CDR3. In certain embodiments, the extracellular domain of the TCR comprises an β chain variable region comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 18 or a conservative modification thereof. SEQ ID NOs: 18, 31, and 32 are disclosed in Table 3. In certain embodiments, the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18.

In certain embodiments, the α chain variable region comprises a CDR1, a CDR2, and a CDR3; and the β chain variable region comprises a CDR1, a CDR2, and a CDR3. In certain embodiments, the α chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 17 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 18 or a conservative modification thereof. In certain embodiments, the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18.

In certain embodiments, the α chain variable region comprises a CDR1, a CDR2, and a CDR3 of the α chain variable region having the amino acid sequence set forth in SEQ ID NO: 19. In certain embodiments, the β chain variable region comprises a CDR1, a CDR2, and a CDR3 of the β chain variable region having the amino acid sequence set forth in SEQ ID NO: 20. In certain embodiments, the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 19. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 19. In certain embodiments, the α chain variable region comprises the amino acid sequence set forth in SEQ ID NO: 19. SEQ ID NO: 19 is provided in Table 3.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 20. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 20. In certain embodiments, the β chain variable region comprises the amino acid sequence set forth in SEQ ID NO: 20. SEQ ID NO: 20 is provided in Table 3.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 19; and the β chain variable region comprises an amino acid sequence that is at least about 80% (e.g., at least about 85%, at least about 90%, or at least about 95%) homologous or identical to the amino acid sequence set forth in SEQ ID NO: 20. In certain embodiments, the α chain variable region comprises the amino acid sequence set forth in SEQ ID NO: 19; and the β chain variable region comprises the amino acid sequence set forth in SEQ ID NO: 20.

In certain embodiments, the TCR is designated as “TCR15”. In certain embodiments, the TCR15 binds to a EWSR1/WT1 peptide comprising or consisting of the amino acid sequence set forth in SEQ ID NO: 4, SEQ ID NO: 5, SEQ ID NO: 6, SEQ ID NO: 7, or SEQ ID NO: 8.

In certain embodiments, the CDRs sequences described above including Table 3 are delineated using the IMGT numbering system.

TABLE 3
(TCR15)

| | |
|---|---|
| CDR1 α-chain | NSAFQY |
| | [SEQ ID NO: 37] |
| CDR2 α-chain | TYSSGN |
| | [SEQ ID NO: 38] |
| CDR3 α-chain | CAMGGGSNYKLTF |
| | [SEQ ID NO: 17] |
| CDR1 ß-chain | SGDLS |
| | [SEQ ID NO: 31] |
| CDR2 ß-chain | YYNGEE |
| | [SEQ ID NO: 32] |
| CDR3 ß-chain | CASSPPGQTNYGYTF |
| | [SEQ ID NO: 18] |
| α-chain | MMKSLRVLLVILWLQLSWVWSQQKEVEQDPGPLSVP |
| variable | EGAIVSLNCTYSNSAFQYFMWYRQYSRKGPELLMYT |
| | YSSGNKEDGRFTAQVDKSSKYISLFIRDSQPSDSAT |
| | YLCAMGGGSNYKLTFGKGTLLTVNP |
| | [SEQ ID NO: 19] |
| ß-chain | MGFRLLCCVAFCLLGAGPVDSGVTQTPKHLITATGQ |
| variable | RVTLRCSPRSGDLSVYWYQQSLDQGLQFLIQYYNGE |
| | ERAKGNILERFSAQQFPDLHSELNLSSLELGDSALY |
| | FCASSPPGQTNYGYTFGSGTRLTVV |
| | [SEQ ID NO: 20] |

In certain embodiments, the extracellular domain of the TCR comprises an α chain variable region comprising a CDR1, a CDR2, and a CDR3. In certain embodiments, the extracellular domain of the TCR comprises an α chain variable region comprising a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof. SEQ ID NOs: 39-41 are disclosed in Table 4. In certain embodiments, the α chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41.

In certain embodiments, the extracellular domain of the TCR comprises an β chain variable region comprising a CDR1, a CDR2, and a CDR3. In certain embodiments, the extracellular domain of the TCR comprises an β chain variable region comprising a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof. SEQ ID NOs: 42-44 are disclosed in Table 4. In certain embodiments, the β chain variable region comprises a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44.

In certain embodiments, the α chain variable region comprises a CDR1, a CDR2, and a CDR3; and the β chain variable region comprises a CDR1, a CDR2, and a CDR3. In certain embodiments, the α chain variable region comprises a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof; and the β chain variable region comprises a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof. In certain embodiments, the α chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41; and the β chain variable region comprises a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44.

In certain embodiments, the α chain variable region comprises a CDR1, a CDR2, and a CDR3 of the α chain variable region having the amino acid sequence set forth in SEQ ID NO: 45. In certain embodiments, the β chain variable region comprises a CDR1, a CDR2, and a CDR3 of the β chain variable region having the amino acid sequence set forth in SEQ ID NO: 46. In certain embodiments, the α chain variable region comprises a CDR1, a CDR2, and a CDR3 of the α chain variable region having the amino acid sequence set forth in SEQ ID NO: 45; and the β chain variable region comprises a CDR1, a CDR2, and a CDR3 of the β chain variable region having the amino acid sequence set forth in SEQ ID NO: 46.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 45. For example, the α chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 45. In certain embodiments, the α chain variable region comprises the amino acid sequence set forth in SEQ ID NO: 45. SEQ ID NO: 45 is provided in Table 4.

In certain embodiments, the β chain variable region comprises an amino acid sequence that is at least about 80% (e.g., at least about 85%, at least about 90%, or at least about 95%) homologous or identical to the amino acid sequence set forth in SEQ ID NO: 46. For example, the β chain variable region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 46. In certain embodiments, the β chain variable region comprises the amino acid sequence set forth in SEQ ID NO: 46. SEQ ID NO: 46 is provided in Table 4.

In certain embodiments, the α chain variable region comprises an amino acid sequence that is at least about 80% (e.g., at least about 85%, at least about 90%, or at least about 95%) homologous or identical to the amino acid sequence set forth in SEQ ID NO: 45; and the β chain variable region comprises an amino acid sequence that is at least about 80% (e.g., at least about 85%, at least about 90%, or at least about 95%) homologous or identical to the amino acid sequence set forth in SEQ ID NO: 46. In certain embodiments, the α chain variable region comprises the amino acid sequence set forth in SEQ ID NO: 45; and the β chain variable region comprises the amino acid sequence set forth in SEQ ID NO: 46.

In certain embodiments, the TCR is designated as “TCR16”. In certain embodiments, the TCR15 binds to a EWSR1/WT1 peptide comprising or consisting of the amino acid sequence set forth in SEQ ID NO: 4, SEQ ID NO: 5, SEQ ID NO: 6, SEQ ID NO: 7, or SEQ ID NO: 8.

In certain embodiments, the CDRs sequences described above including Table 4 are delineated using the IMGT numbering system.

TABLE 4
(TCR16)

| | |
|---|---|
| CDR1 α-chain | VSPFSN |
| | [SEQ ID NO: 39] |
| CDR2 α-chain | MTFSENT |
| | [SEQ ID NO: 40] |
| CDR3 α-chain | CVVSAARGSTLGRLYF |
| | [SEQ ID NO: 41] |
| CDR1 ß-chain | SNHLY |
| | [SEQ ID NO: 42] |
| CDR2 ß-chain | FYNNEI |
| | [SEQ ID NO: 43] |
| CDR3 ß-chain | CASSLGQSSSYNSPLHF |
| | [SEQ ID NO: 44] |
| α-chain | MKKHLTTFLVILWLYFYRGNGKNQVEQSPQSLIILE |
| variable | GKNCTLQCNYTVSPFSNLRWYKQDTGRGPVSLTIMT |
| | FSENTKSNGRYTATLDADTKQSSLHITASQLSDSAS |
| | YICVVSAARGSTLGRLYFGRGTQLTVWP |
| | [SEQ ID NO: 45] |
| ß-chain | MDTWLVCWAIFSLLKAGLTEPEVTQTPSHQVTQMGQ |
| variable | EVILRCVPISNHLYFYWYRQILGQKVEFLVSFYNNE |
| | ISEKSEIFDDQFSVERPDGSNFTLKIRSTKLEDSAM |
| | YFCASSLGQSSSYNSPLHFGNGTRLTVT |
| | [SEQ ID NO: 46] |

In certain embodiments, the α chain variable region and/or the β chain variable region amino acid sequences have at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences (e.g., SEQ ID NO: 11, SEQ ID NO: 12, SEQ ID NO: 15, SEQ ID NO: 16, SEQ ID NO: 19, SEQ ID NO: 20, SEQ ID NO: 45, and SEQ ID NO: 46) comprise modifications, including, but not limited to, substitutions (e.g., conservative substitutions), insertions, or deletions relative to the specified sequence(s), but retain the ability to bind to a EWSR1/WT1 peptide. In certain embodiments, such modifications are not within the CDR domains of the variable regions.

In certain embodiments, a total of 1 to 10 amino acids are substituted, inserted and/or deleted in SEQ ID NO: 11, SEQ ID NO: 12, SEQ ID NO: 15, SEQ ID NO: 16, SEQ ID NO: 19, SEQ ID NO: 20, SEQ ID NO: 45, or SEQ ID NO: 46. In certain embodiments, substitutions, insertions, or deletions occur in regions outside the CDRs of the extracellular domain. In certain embodiments, the extracellular domain comprises an α chain variable region and/or a β chain variable region sequence selected from the group consisting of SEQ ID NO: 11, SEQ ID NO: 12, SEQ ID NO: 15, SEQ ID NO: 16, SEQ ID NO: 19, SEQ ID NO: 20, SEQ ID NO: 45, or SEQ ID NO: 46 including post-translational modifications of that sequence (SEQ ID NO: 11, SEQ ID NO: 12, SEQ ID NO: 15, SEQ ID NO: 16, SEQ ID NO: 19, SEQ ID NO: 20, SEQ ID NO: 45, or SEQ ID NO: 46).

### 5.3.1.2. Constant Regions

In certain embodiments, the presently disclosed TCR comprises an α chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 21 or SEQ ID NO: 22. In certain embodiments, the α chain constant region comprises the amino acid sequence set forth in SEQ ID NO: 21. In certain embodiments, the α chain constant region comprises the amino acid sequence set forth in SEQ ID NO: 22.

In certain embodiments, the presently disclosed TCR comprises a β chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23, SEQ ID NO: 24, SEQ ID NO: 25, or SEQ ID NO: 26. In certain embodiments, the β chain constant region comprises the amino acid sequence set forth in SEQ ID NO: 23. In certain embodiments, the β chain constant region comprises the amino acid sequence set forth in SEQ ID NO: 24. In certain embodiments, the β chain constant region comprises the amino acid sequence set forth in SEQ ID NO: 25. In certain embodiments, the β chain constant region comprises the amino acid sequence set forth in SEQ ID NO: 26. SEQ ID NOS: 21-26 are provided below:

Human α chain constant region:

[SEQ ID NO: 21]
NIQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKLV
EKSFETDTNLNFQNLSVIGFRILLLKVAGFNLLMTLRLWSS

Mouse α chain constant region (cysteine-modification and LVL modification in transmembrane domain underlined):

[SEQ ID NO: 22]
NIQNPEPAVYQLKDPRSQDSTLCLFTDFDSQINVPKTMESGTFITDKCVL
DMKAMDSKSNGAIAWSNQTSFTCQDIFKETNATYPSSDVPCDATLTEKSF
ETDMNLNFQNLLVIVLRILLLKVAGFNLLMTLRLWSS

Human β chain constant region:

[SEQ ID NO: 23]
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF

Human β chain constant region:

[SEQ ID NO: 24]
EDLKNVFPPKVAVFEPSEAEISHTQKATLVCLATGFYPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSESYQQGVLSATILYE
ILLGKATLYAVLVSALVLMAMVKRKDSRG

Human β chain constant region:

[SEQ ID NO: 25]
EDLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSESYQQGVLSATILYE
ILLGKATLYAVLVSALVLMAMVKRKDSRG

Mouse β chain constant region (cysteine-modification underlined):

[SEQ ID NO: 26]
EDLRNVTPPKVSLFEPSKAEIANKQKATLVCLARGFFPDHVELSWWVNGK
EVHSGVCTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS

5.3.2. TCRs that Bind to the Same EWSR1/WT1 Peptide as TCR Clonotypes

The presently disclosed subject matter further provides TCRs that bind to the same EWSR1/WT1 peptide comprising the junctional amino acid sequence of the fusion protein as a TCR disclosed herein (e.g., a TCR disclosed in Section 5.3.1). In certain embodiments, the TCR binds to the same EWSR1/WT1 peptide comprising the junctional amino acid sequence of the fusion protein as a reference TCR or a functional fragment thereof comprising the α chain variable region CDR1, CDR2, and CDR3 sequences and the β chain variable region CDR1, CDR2, and CDR3 sequences of, for example, any one of the TCRs disclosed herein (e.g., those disclosed in Section 5.3.1). In certain embodiments, the TCR binds to the same EWSR1/WT1 peptide comprising the junctional amino acid sequence of the fusion protein as a reference TCR or a functional fragment thereof comprising the α chain variable region and the β chain variable region sequences of, for example, any one of the presently disclosed TCRs (e.g., those disclosed in Section 5.3.1).

### 5.3.3. TCRs Having Specific CDR3 Sequences

It is well known in the art that the CDR3 domain, independently from the CDR1 and/or CDR2 domain(s), alone can determine the binding specificity of a TCR or a functional fragment thereof, for a cognate antigen and that multiple TCRs can predictably be generated having the same binding specificity based on a common CDR3 sequence.

In certain embodiments, the extracellular domain of the TCR comprises an α chain variable region CDR3 comprising the amino acid sequence set forth in SEQ ID NO: 9 or a conservative modification thereof; and a β chain variable region CDR3 comprising the amino acid sequence set forth in SEQ ID NO: 10 or a conservative modification thereof.

In certain embodiments, the extracellular domain of the TCR comprises an α chain variable region CDR3 comprising the amino acid sequence set forth in SEQ ID NO: 13 or a conservative modification thereof; and a β chain variable region CDR3 comprising the amino acid sequence set forth in SEQ ID NO: 14 or a conservative modification thereof.

In certain embodiments, the extracellular domain of the TCR comprises an α chain variable region CDR3 comprising the amino acid sequence set forth in SEQ ID NO: 17 or a conservative modification thereof; and a β chain variable region CDR3 comprising the amino acid sequence set forth in SEQ ID NO: 18 or a conservative modification thereof.

In certain embodiments, the extracellular domain of the TCR comprises an α chain variable region CDR3 comprising the amino acid sequence set forth in SEQ ID NO: 41 or a conservative modification thereof; and a β chain variable region CDR3 comprising the amino acid sequence set forth in SEQ ID NO: 44 or a conservative modification thereof.

5.3.4. TCRs with Modifications within CDRs

In certain embodiments, a presently disclosed TCR (or a functional fragment thereof) comprises an α chain variable region comprising CDR1, CDR2 and CDR3 sequences and a β chain variable region comprising CDR1, CDR2 and CDR3 sequences, wherein one or more of these CDR sequences comprise specified amino acid sequences based on the TCRs (or a functional fragments thereof) described herein (see Tables 1-4), or modifications thereof, and wherein the TCRs (or a functional fragments thereof) retain the desired functional properties of the EWSR1/WT1 peptide-specific TCRs (or a functional fragments thereof) of the presently disclosed subject matter.

In certain embodiments, a presently disclosed TCR (or a functional fragment thereof) comprises an α chain constant region and a β chain constant region, wherein at least one of the constant regions comprises specified amino acid sequences based on the TCRs (or a functional fragments thereof) described herein (see Tables 1-4), or modifications thereof, and wherein the TCR (or a functional fragment thereof) retains the desired functional properties of the EWSR1/WT1 peptide-specific TCRs (or a functional fragments thereof) of the presently disclosed subject matter.

In certain embodiments, such modifications do not significantly affect or alter the binding characteristics of the TCR comprising the amino acid sequence. Non-limiting examples of such modifications include amino acid substitutions, additions and deletions. Modifications can be introduced into the presently disclosed TCR or a functional fragment thereof by standard techniques known in the art, such as site-directed mutagenesis and PCR-mediated mutagenesis.

The modifications can be conservative modifications, non-conservative modifications, or mixtures of conservative and non-conservative modifications. As discussed above, conservative amino acid substitutions are ones in which the amino acid residue is replaced with an amino acid residue having a similar side chain. Families of amino acid residues having similar side chains have been defined in the art. Exemplary conservative amino acid substitutions are shown in Table 5 below. In certain embodiments, amino acid substitutions may be introduced into a TCR of interest and the products screened for a desired activity, e.g., retained/improved antigen binding, decreased immunogenicity, or improved ADCC or CDC.

TABLE 5

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

In certain embodiments, one or more amino acid residues within a CDR region can be replaced with other amino acid residues from the same group and the altered TCR can be tested for retained function using the functional assays described herein.

Non-conservative substitutions entail exchanging a member of one of these classes for another class.

In certain embodiments, no more than one, no more than two, no more than three, no more than four, no more than five residues within a specified sequence or a CDR region are altered.

In certain embodiments, one or more amino acid residues within a constant region of a TCR can be modified to enhance stability and/or cell surface expression of the TCR. In certain embodiments, no more than one, no more than two, no more than three, no more than four, no more than five residues within a specified sequence or a constant region are altered. In certain embodiments, the modification includes but is not limited to, murinization, cysteine modification and transmembrane modification (see Cohen et al. Enhanced antitumor activity of murine-human hybrid T-cell receptor (TCR) in human lymphocytes is associated with improved pairing and TCR/CD3 stability, Cancer Res. 2006; 66 (17): 8878-8886; Cohen et al. Enhanced antitumor activity of T cells engineered to express T-cell receptors with a second disulfide bond, Cancer Res. 2007; 67 (8): 3898-3903; Kuball et al. Facilitating matched pairing and expression of TCR chains introduced into human T cells, Blood 2007; 109 (6): 2331-2338; Haga-Friedman et al. Incorporation of transmembrane hydrophobic mutations in the TCR enhance its surface expression and T cell functional avidity, Journal of immunology 2012; 188 (11): 5538-5546, the contents of each of which are incorporated by reference in their entireties).

### 5.3.5. Multispecific Molecules

The presently disclosed subject matter provides multispecific molecules comprising a presently disclosed TCR (or a functional fragment thereof). A presently disclosed TCR or a functional fragment thereof can be derivatized or linked to one or more functional molecules, e.g., one or more peptides or proteins (e.g., one or more antibodies or ligands for a receptor) to generate a multispecific molecule that binds to at least two different binding sites and/or target molecules. To create a multispecific molecule, a presently disclosed TCR or a functional fragment thereof can be functionally linked (e.g., by chemical coupling, genetic fusion, noncovalent association or otherwise) to one or more other binding molecules, such as one or more antibodies, antibody fragments, peptides or binding mimetic.

The presently disclosed subject matter provides multispecific molecules comprising at least a first binding specificity for a EWSR1/WT1 peptide and a second binding specificity for a second target peptide region. The second target epitope region can be a second EWSR1/WT1 peptide, or a non-EWSR1/WT1 peptide, e.g., a different antigen. In certain embodiments, the multi-specific molecule further comprises a third binding specificity. Where a first portion of a multispecific molecule, e.g., a presently disclosed TCR, binds to an antigen on a tumor cell for example and a second portion of a multispecific molecule recognizes an antigen on the surface of a human immune effector cell, the multispecific molecule is capable of recruiting the activity of that effector cell by specifically binding to the effector antigen on the human immune effector cell. In certain embodiments, multispecific molecules are able to form a link between effector cells, for example, T cells and tumor cells, thereby enhancing effector function. In certain embodiments, a presently disclosed multispecific molecule comprises at least a first binding to a EWSR1/WT1 peptide and at least a second binding to an immune cell or a molecule associated with an immune cell.

The multispecific molecules of the presently disclosed subject matter can be prepared by conjugating the constituent binding specificities using methods known in the art. For example, each binding specificity of the multispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio) propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multispecific molecule comprises a monoclonal antibody (mAb) and a mAb, a mAb and a Fab, a Fab and a F (ab′) 2, or a ligand and a Fab fusion protein.

Binding of the multispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography.

### 5.4. Cells

The presently disclosed subject matter provides cells comprising a presently disclosed TCR (e.g., one disclosed in Section 5.3). In certain embodiments, the TCR is recombinant. In certain embodiments, the TCR is exogenous. In certain embodiments, the TCR is recombinant and/or exogenous. In certain embodiments, the cell is selected from the group consisting of cells of lymphoid lineage, and stem cells from which cells of lymphoid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, B cells, Natural Killer cells, and stem cells from which lymphoid cells may be differentiated. In certain embodiments, the stem cell is a pluripotent stem cell (e.g., embryonic stem cell, induced pluripotent stem cell (iPSC)).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8+ T cell. In certain embodiments, the T cell is a CD4⁺ T cell. In certain embodiments, the T cell is a CD8⁺ T cell. In certain embodiments, the T cell is a γδ T cell. In certain embodiments, the T cell is a Natural killer T cell. In certain embodiments, the TCR-expressing T cells express Foxp3 to achieve and maintain a T regulatory phenotype.

In certain embodiments, the cell is a Natural Killer cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

In certain embodiments, the cell further comprises at least one recombinant or exogenous co-stimulatory ligand. For example, a presently disclosed cell can be further transduced with at least one co-stimulatory ligand, such that the cell co-expresses or is induced to co-express the presently disclosed TCR and the at least one co-stimulatory ligand. The interaction between the presently disclosed TCR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNF-β)/lymphotoxin-alpha (LTa), lymphotoxin-beta (LTB), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the cell comprises one recombinant co-stimulatory ligand that is 4-1BBL. In certain embodiments, the cell comprises two recombinant co-stimulatory ligands that are 4-1BBL and CD80.

In certain embodiments, a presently disclosed cell further comprises at least one exogenous cytokine. For example, a presently disclosed cell can be further transduced with at least one cytokine, such that the cell secretes the at least one cytokine as well as expresses the presently disclosed TCR. In certain embodiments, the at least one cytokine is selected from the group consisting of IL-2, IL-3, IL-6, IL-7, IL-11, IL-12, IL-15, IL-17, IL-18, and IL-21. In certain embodiments, the cytokine is IL-12.

In certain embodiments, a presently disclosed cell further comprises at least one exogenous co-receptor. For example, a presently disclosed cell can be further transduced with at least one co-receptor, such that the cell co-expresses or is induced to co-express the presently disclosed TCR and the at least one co-receptor. In certain embodiments, the at least one co-receptor is selected from the group consisting of a CD45 co-receptor, a CD8 co-receptor, and a CD4 co-receptor. In certain embodiments, the at least one co-receptor comprises a CD8 co-receptor.

In certain embodiments, the CD8 co-receptor comprises an α chain and a β chain. In certain embodiments, the α chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P01732, or fragments thereof, and/or may optionally comprise up to one or up to two or up to three conservative amino acid substitutions. In certain embodiments, the α chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 48. In certain embodiments, the α chain of the CD8 co-receptor comprises or consists of the amino acid sequence set forth in SEQ ID NO: 48.

In certain embodiments, the β chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P10966, or fragments thereof, and/or may optionally comprise up to one or up to two or up to three conservative amino acid substitutions. In certain embodiments, the β chain of the CD8 co-receptor comprises or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 49. In certain embodiments, the β chain of the CD8 co-receptor comprises or consists of the amino acid sequence set forth in SEQ ID NO: 49.

In certain embodiments, the CD8 co-receptor comprises an α chain comprising or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 48; and a β chain comprising or consists of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 49. In certain embodiments, the CD8 co-receptor comprises an α chain comprising or consists of the amino acid sequence set forth in SEQ ID NO: 48; and a β chain comprising or consists of the amino acid sequence set forth in SEQ ID NO: 49. SEQ ID NO: 48 and SEQ ID NO: 49 are provided below:

[SEQ ID NO: 48]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV
[SEQ ID NO: 49]
MRPRLWLLLAAQLTVLHGNSVLQQTPAYIKVQTNKMVMLSCEAKISLSNM
RIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTL
KKRVCRLPRPETQKGPLCSPITLGLLVAGVLVLLVSLGVAIHLCCRRRRA
RLRFMKQFYK

In certain embodiments, the co-receptor is a CD4 co-receptor. In certain embodiments, the CD4 co-receptor comprises a polypeptide comprising or consisting of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence having a UniProt Reference No: P01730, or fragments thereof, and/or may optionally comprise up to one or up to two or up to three conservative amino acid substitutions. In certain embodiments, the CD4 co-receptor comprises a polypeptide comprising or consisting of an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 50. In certain embodiments, the CD4 co-receptor comprises a polypeptide comprising or consisting of the amino acid sequence set forth in SEQ ID NO: 50. SEQ ID NO: 50 is provided below:

[SEQ ID NO: 50]
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVEGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI

In certain embodiments, a presently disclosed cell further comprises at least one exogenous integrin. For example, a presently disclosed cell can be further transduced with at least one integrin, such that the cell co-expresses or is induced to co-express the presently disclosed TCR and the at least one integrin. In certain embodiments, the at least one integrins is selected from the group consisting of LFA-1 and VLA-4. In certain embodiments, the at least one integrin comprises LFA-1.

### 5.5. Nucleic Acids and Genetic Modifications of Cells

The present discloses subject matter provides a nucleic acid encoding a presently disclosed TCR (e.g., one disclosed in Section 5.3). Further provided are cells comprising such nucleic acids. In certain embodiments, a promoter is operably linked to the presently disclosed TCR.

In certain embodiments, the promoter is endogenous or exogenous. In certain embodiments, the exogenous promoter is selected from the group consisting of a long terminal repeat (LTR) promoter, an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiment, the exogenous promoter is a LTR promoter. In certain embodiments, the promoter is an inducible promoter. In certain embodiment, the inducible promoter is selected from the group consisting of a NFAT transcriptional response element (TRE) promoter, a CD69 promoter, a CD25 promoter, and an IL-2 promoter.

In certain embodiments, the nucleic acid encodes both the α chain and the β chain of a presently disclosed TCR. In certain embodiments, the α chain and the β chain are separated by a self-cleavage peptide, e.g., a 2A-peptide. In certain embodiments, the α chain and the β chain are separated by a furin-2A-peptide. In certain embodiments, the peptide comprises the amino acid sequence set forth in SEQ ID NO: 27.

[SEQ ID NO: 27]
RAKRSGSGATNFSLLKQAGDVEENPGP

In certain embodiments, the nucleic acid encodes a functional portion/fragment of a presently disclosed TCR. As used herein, the term “functional portion” or “functional fragment” refers to any portion, part or fragment of a presently disclosed TCR, which portion, part or fragment retains the biological activity of the TCR (the parent TCR). For example, functional portions encompass the portions, parts or fragments of a presently disclosed TCR that retains the ability to recognize the EWSR1/WT1 peptide to a similar, same, or even a higher extent as the parent TCR. In certain embodiments, the nucleic acid encoding a functional portion of a presently disclosed TCR encodes a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

Genetic modification of a cell (e.g., a T cell) can be accomplished by transducing a substantially homogeneous cell composition with a recombinant DNA or RNA construct. In certain embodiments, a retroviral vector (e.g., gamma-retroviral vector or lentiviral vector) is employed for the introduction of the DNA or RNA construct into the cell. For example, a polynucleotide encoding a presently disclosed TCR can be cloned into a retroviral vector and expression can be driven from its endogenous promoter, from the retroviral long terminal repeat, or from an alternative internal promoter, or from a promoter specific for a target cell type of interest. Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used. For initial genetic modification of a cell to include a presently disclosed TCR, a retroviral vector can be employed for transduction, however any other suitable viral vector or non-viral delivery system can be used. The TCR can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107: 77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell(s) (or its descendants) are injected into a targeted tissue or are injected systemically. Recombinant receptors can also be derived or obtained using transposases or targeted nucleases (e.g., Zinc finger nucleases, meganucleases, or TALE nucleases, CRISPR). Transient expression may be obtained by RNA electroporation.

In certain embodiments, a presently disclosed TCR can be integrated into a selected locus of the genome of a cell. Any targeted genome editing methods can also be used to deliver a presently disclosed TCR to a cell or a subject. In certain embodiments, a CRISPR system is used to deliver a presently disclosed TCR. In certain embodiments, zinc-finger nucleases are used to deliver presently disclosed TCR. In certain embodiments, a TALEN system is used to deliver a presently disclosed TCR.

In certain embodiments, a presently disclosed TCR can be integrated at a locus encoding a T cell receptor. Non-limiting examples of the loci include a TRAC locus, a TRBC locus, a TRDC locus, and a TRGC locus. In certain embodiments, the locus is a TRAC locus or a TRBC locus. Methods of targeting a TCR to a site within the genome of T cell can be found in WO2017180989 and Eyquem et al., Nature. (2017 Mar. 2); 543 (7643): 113-117, both of which are incorporated by reference in their entireties.

In certain embodiments, a presently disclosed TCR can be integrated at a genetic locus encoding an immune checkpoint. Non-limiting examples of the loci include a PDCD1 locus, a CBLB locus, a CISH locus, or a RASA2 locus. In certain embodiments, the locus is a PDCD1 locus. In certain embodiments, the locus is a CBLB locus. In certain embodiments, the locus is a CISH locus. In certain embodiments, the locus is a RASA2 locus. Non-limiting examples of methods of integrating a presently disclosed TCR to a locus encoding an immune-checkpoint CRISPR systems, zinc-finger nucleases, and TALEN systems.

In certain embodiments, a presently disclosed TCR can be integrated at a genomic safe harbor locus. As used herein, a “genomic safe harbor” or “GSH” refers to a chromosome location where an integrated transgene (e.g., encoding a presently disclosed TCR) can be predictably expressed without adversely affecting endogenous gene structure or expression. In certain embodiments, integrating a transgene at the GSH does not alter cell behavior and/or promote malignant transformation of the host cell or the organism. In certain embodiments, the GSH permits sufficient transgene expression to yield desirable levels of protein or non-coding RNA encoded by the transgene. Additional information on genomic safe harbor sites can be found in International Patent Publication No. WO 2021/055616, Sadelain et al., Nature Reviews Cancer 12.1 (2012): 51-58, and Aznauryan et al., Cell Reports Methods 2.1 (2022), the contents of each of which are incorporated by reference in their entirety.

In certain embodiments, the expression of the TCR is driven by an endogenous promoter/enhancer within or near the locus. In certain embodiments, the expression of the TCR is driven by an exogenous promoter integrated into the locus. The locus where the TCR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the TCR in selected loci of the genome of a cell. In certain embodiments, the CRISPR system uses a DNA donor-template guided homology directed repair at a defined genetic locus, e.g., a TRAC locus. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. In certain embodiments, CRISPR/Cas9 is a recombinant ribonucleoprotein complex that is transfected into target cells. The crRNA needs to be designed for each application as this is the sequence that Cas9 uses to identify and directly bind to the target DNA in a cell. The repair template carrying TCR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the TCR in selected loci of the genome of a cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying TCR expression cassette, ZFNs can be used to insert the TCR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the TCR in selected loci of the genome of an immunoresponsive cell. Transcription activator-like effector nucleases (TALEN) are restriction enzymes that can be engineered to cut specific sequences of DNA. TALEN system operates on almost the same principle as ZENs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include the use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10-9 to about 10-6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include the use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes, so the contents are transferred into the cytoplasm.

In certain embodiments, the delivery methods include the use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″-(((methylazanediyl)bis(propane-3,1 diyl))bis (azanetriyl))tetrapropionate; decyl (2-(dioctylammonio)ethyl) phosphate; ((4-hydroxybutyl) azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl)bis(dodecan-2-ol); cKK-E12, 3,6-bis(4-(bis(2-hydroxydodecyl)amino)butyl) piperazine-2,5-dione; (6Z,9Z,28Z,31Z)-heptatriaconta-6,9,28,31-tetraen-19-yl 4-(dimethylamino) butanoate; hexa (octan-3-yl) 9,9′,9″,9″,9″″,9″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9 ‘Z,9=Z,9″Z,12Z,12’ Z,12″ Z,12″Z)-tetrakis(octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating their integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

Modification can be made anywhere within the selected locus, or anywhere that can influence gene expression of the integrated TCR. In certain embodiments, the modification is introduced upstream of the transcriptional start site of the integrated TCR. In certain embodiments, the modification is introduced between the transcriptional start site and the protein coding region of the integrated TCR) In certain embodiments, the modification is introduced downstream of the protein coding region of the integrated TCR.

### 5.6. Formulations and Administration

The presently disclosed subject matter also provides compositions comprising the presently disclosed cells (e.g., those disclosed in Section 5.4). In certain embodiments, the composition is a pharmaceutical composition that further comprises a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a tumor. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹¹, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, at least about 1× 10⁵ cells can be administered, eventually reaching about 1×10¹⁰ or more. In certain embodiments, at least about 1×10⁶ cells can be administered. In certain embodiments, from about 10⁴ to about 10¹¹, from about 10⁵ to about 10⁹, or from about 10⁶ to about 10⁸ the presently disclosed cells are administered to a subject. More effective cells may be administered in even smaller numbers. In certain embodiments, at least about 1×10⁸, about 2×10⁸, about 3×10⁸, about 4×10⁸, and about 5× 10⁸ the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5.7. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for prolonging the survival of a subject suffering from a tumor.

In certain embodiments, each of the above-noted methods comprises administering the presently disclosed cells or a composition (e.g., a pharmaceutical composition) comprising thereof to achieve the desired effect, e.g., palliation of an existing condition or prevention of recurrence of tumor. For treatment, the amount administered is an amount effective in producing the desired effect. An effective amount can be provided in one or a series of administrations. An effective amount can be provided in a bolus or by continuous perfusion.

In certain embodiments, the tumor is associated with a EWSR1/WT1 fusion protein.

In certain embodiments, the tumor is a cancer. In certain embodiments, the tumor is selected from the group consisting of desmoplastic small round cell tumor (DSRCT), soft tissue sarcoma, colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer (also known as “stomach cancer”), neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma. In certain embodiments, the tumor is desmoplastic small round cell tumor (DSRCT).

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In certain embodiments, the subject comprises an HLA-A. In certain embodiments, the HLA-A is an HLA-A*03 superfamily member. In certain embodiments, the HLA-A*03 superfamily member is selected from the group consisting of HLA-A*03, HLA-A*11, HLA-A*33, HLA-A*66, HLA-A*68, and HLA-A*74. In certain embodiments, the subject comprises an HLA-A*03 molecule. In certain embodiments, the HLA-A*03 molecule is selected from the group consisting of HLA-A*0301, HLA-A*0302, and HLA-A*0305.

In certain embodiments, the subject comprises an HLA-A. In certain embodiments, the subject comprises an HLA-A*11 molecule. In certain embodiments, the HLA-A*11 molecule is selected from the group consisting of HLA-A*1101, HLA-A*1102, HLA-A*1104, and HLA-A*1105.

### 5.8. Diagnostic and Prognostic Methods

The presently disclosed TCR, multi-specific molecules, and nucleic acids encode thereof can be used for diagnostic and prognostic applications as well as use as research tools for detection of the EWSR1/WT1 peptide in a biological sample, in a cell, a tissue, or a blood sample. The presently disclosed subject matter provides methods for detecting the EWSR1/WT1 peptide in a cell, a tissue, or a blood sample. In certain embodiments, the method comprises: contacting a cell, a tissue, or a blood sample with presently disclosed TCR (or a functional fragment thereof) or multi-specific molecule disclosed herein, wherein the TCR or multi-specific molecule comprises a detectable label; and determining the amount of the labeled TCR or multi-specific molecule bound to the cell, tissue, or blood sample by measuring the amount of detectable label associated with the cell or tissue, wherein the amount of bound TCR or multi-specific molecule indicates the amount of the EWSR1/WT1 peptide in the cell, tissue, or a blood sample. The cell or tissue can be any cell or tissue, including any normal, healthy, or cancerous cells and tissues. In certain embodiments, the blood sample is a peripheral blood sample.

The presently disclosed TCR (or a functional fragment thereof) can be used in methods known in the art relating to the localization and/or quantitation of the EWSR1/WT1 peptide (e.g., for use in measuring levels of the EWSR1/WT1 peptide within appropriate physiological samples, for use in diagnostic methods, for use in imaging the peptide, and the like). The presently disclosed TCR (or a functional fragment thereof) can be used to isolate a cell including a EWSR1/WT1 peptide by standard techniques, such as affinity chromatography or immunoprecipitation. Moreover, the presently disclosed TCR (or a functional fragment thereof) can be used to detect an immunoreactive EWSR1/WT1 peptide (e.g., in plasma, a cellular lysate or cell supernatant) in order to evaluate the abundance and pattern of expression of the immunoreactive polypeptide. the presently disclosed TCR (or a functional fragment thereof) can be used diagnostically to monitor immunoreactive EWSR1/WT1 peptide levels in tissue as part of a clinical testing procedure, e.g., to determine the efficacy of a given treatment regimen. As noted above, the detection can be facilitated by coupling (i.e., physically linking) the presently disclosed TCR (or a functional fragment thereof) to a detectable substance.

An exemplary method for detecting the presence or absence of an immunoreactive EWSR1/WT1 peptide in a biological sample comprises contacting a biological sample from a subject with the presently disclosed TCR (or a functional fragment thereof), wherein the presence of an immunoreactive EWSR1/WT1 peptide is detected in the biological sample. Detection may be accomplished by means of a detectable label attached to the antibody.

The term “labeled” with regard to the presently disclosed TCR (or a functional fragment thereof) is intended to encompass direct labeling of the TCR by coupling (i.e., physically linking) a detectable substance to the antibody, as well as indirect labeling of the antibody by reactivity with another compound that is directly labeled, such as a secondary antibody.

In certain embodiments, the presently disclosed TCR (or a functional fragment thereof) is conjugated to one or more detectable labels. For such uses, the presently disclosed TCR (or a functional fragment thereof) may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive EWSR1/WT1 peptide in a biological sample in vitro as well as in vivo. Non-limiting examples of in vitro techniques for detection of an immunoreactive EWSR1/WT1 peptide include enzyme linked immunosorbent assays (ELISAs), Western blots, immunoprecipitations, radioimmunoassay, and immunofluorescence. Furthermore, in vivo techniques for detection of an immunoreactive EWSR1/WT1 peptide include introducing into a subject a labeled TCR (or a functional fragment thereof). For example, the presently disclosed TCR (or a functional fragment thereof) can be labeled with a radioactive marker whose presence and location in a subject can be detected by standard imaging techniques. In certain embodiments, the biological sample comprises EWSR1/WT1 peptide molecules from the test subject.

The presently disclosed TCR (or a functional fragment thereof) can be used to assay immunoreactive EWSR1/WT1 peptide levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive EWSR1/WT1 peptide levels in a biological sample, the presently disclosed TCR (or a functional fragment thereof) may be used for in vivo imaging of EWSR1/WT1 peptide. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the presently disclosed TCR (or a functional fragment thereof).

The presently disclosed TCR (or a functional fragment thereof), which are labeled with an appropriate detectable imaging moiety (such as a radioisotope (e.g., ¹³¹I, ¹¹¹In, ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries. The labeled TCR (or a functional fragment thereof) then accumulates at the location of cells which contain the specific target polypeptide. For example, the presently disclosed TCR (or a functional fragment thereof) accumulate within the subject in cells and tissues in which the EWSR1/WT1 peptide has localized.

Thus, the presently disclosed subject matter provides diagnostic methods of a medical condition. In certain embodiments, the method comprises: (a) assaying the expression of immunoreactive EWSR1/WT1 peptide by measuring binding of a presently disclosed TCR (or a functional fragment thereof) in cells or body fluid of an individual; and (b) comparing the amount of immunoreactive EWSR1/WT1 peptide present in the sample with a standard reference, wherein an increase or decrease in immunoreactive EWSR1/WT1 peptide levels compared to the standard is indicative of a medical condition.

Furthermore, the presently disclosed TCR (or a functional fragment thereof) may be used to purify cells including a EWSR1/WT1 peptide from a sample. In certain embodiments, the TCRs are immobilized on a solid support. Non-limiting examples of such solid supports include plastics such as polycarbonate, complex carbohydrates such as agarose and sepharose, acrylic resins and such as polyacrylamide and latex beads. Techniques for coupling TCRs to such solid supports are well known in the art.

A TCR or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl) propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

A TCR or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high-density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. A TCR or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed TCR (or a functional fragment thereof) is useful in diagnostic methods. As such, the presently disclosed subject matter provides methods using the presently disclosed TCR (or a functional fragment thereof) in diagnosis of EWSR1/WT1 peptide activity in a subject. The presently disclosed TCR (or a functional fragment thereof) may be selected such that they have any level of epitope binding specificity and high binding affinity to a EWSR1/WT1 peptide.

The presently disclosed TCR (or a functional fragment thereof) can be used to detect an immunoreactive EWSR1/WT1 peptide in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of EWSR1/WT1 peptide in a sample obtained from the subject. In certain embodiments, the sample is selected from the group consisting of urine, blood, serum, plasma, saliva, amniotic fluid, cerebrospinal fluid (CSF), and biopsied body tissue.

In certain embodiments, the presently disclosed TCR (or a functional fragment thereof) is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed TCR (or a functional fragment thereof) using standard chemistries. The chelate is normally linked to the antibody by a group which enables formation of a bond to the molecule with minimal loss of immunoreactivity and minimal aggregation and/or internal cross-linking.

### 5.9. Kits

The presently disclosed subject matter provides kits for treatment or ameliorating a disease or disorder associated with EWSR1/WT1 peptide (e.g., a cancer cell), and/or detecting EWSR1/WT1 peptide. In certain embodiments, the kit comprises the presently disclosed TCR (or a functional fragment thereof), the cells, the multi-specific molecule, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the presently disclosed TCR (or a functional fragment thereof), the cells, the multi-specific molecule, or the composition disclosed herein to a subject in need the treatment. The instructions can generally include information about the use of the presently disclosed TCR (or a functional fragment thereof), the cells, the multi-specific molecule, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

Embodiment 1. A recombinant T cell receptor (TCR) that binds to an EWSR1/WT1 peptide.

Embodiment 2. The recombinant TCR of embodiment 1, wherein the EWSR1/WT1 peptide comprises a junctional amino acid sequence of the fusion protein between EWS and WT1.

Embodiment 3. The recombinant TCR of embodiment 1 or 2, wherein the EWSR1/WT1 peptide comprises or consists of the amino acid sequence set forth in SEQ ID NO: 3, SEQ ID NO: 4, SEQ ID NO: 5, SEQ ID NO: 6, SEQ ID NO: 7, or SEQ ID NO: 8.

Embodiment 4. The recombinant TCR of any one of embodiments 1-3, wherein the EWSR1/WT1 peptide is associated with an HLA class I complex.

Embodiment 5. The recombinant TCR of embodiment 4, wherein the HLA class I complex is selected from an HLA-A, an HLA-B, and an HLA-C.

Embodiment 6. The recombinant TCR of embodiment 4 or 5, wherein the HLA class I complex is an HLA-A.

Embodiment 7. The recombinant TCR of embodiment 4 or 5, wherein the HLA-A is an HLA-A*03 superfamily member.

Embodiment 8. The recombinant TCR of embodiment 7, wherein the HLA-A*03 superfamily member is selected from the group consisting of HLA-A*03, HLA-A*11, HLA-A*33, HLA-A*66, HLA-A*68, and HLA-A*74.

Embodiment 9. The recombinant TCR of embodiment 7 or 8, wherein the HLA-A*03 superfamily member is HLA-A*03.

Embodiment 10. The recombinant TCR of embodiment 9, wherein the HLA-A*03 molecule is selected from the group consisting of HLA-A*0301, HLA-A*0302, and HLA-A*0305.

Embodiment 11. The recombinant TCR of embodiment 7 or 8, wherein the HLA-A*03 superfamily member is HLA-A*11.

Embodiment 12. The recombinant TCR of embodiment 11, wherein the HLA-A*11 molecule is selected from the group consisting of HLA-A*1101, HLA-A*1102, HLA-A*1104, and HLA-A*1105.

Embodiment 13. The recombinant TCR of any one of embodiments 1-12, wherein the TCR comprises an extracellular domain that binds to the EWSR1/WT1 peptide, wherein the extracellular domain comprises an α chain and a β chain, wherein the α chain comprises an α chain variable region and a chain constant region, and the β chain comprises a β chain variable region and a β chain constant region.

Embodiment 14. The recombinant TCR of embodiment 13, wherein:
a) the α chain variable region comprises a CDR3 comprising the amino acid sequence set forth in SEQ ID NO: 9 or a conservative modification thereof, and the β chain variable region comprises a CDR3 comprising the amino acid sequence set forth in SEQ ID NO: 10 or a conservative modification thereof;
b) the α chain variable region comprises a CDR3 comprising the amino acid sequence set forth in SEQ ID NO: 13 or a conservative modification thereof, and the β chain variable region comprises a CDR3 comprising the amino acid sequence set forth in SEQ ID NO: 14 or a conservative modification thereof;
c) the α chain variable region comprises a CDR3 comprising the amino acid sequence set forth in SEQ ID NO: 17 or a conservative modification thereof, and the β chain variable region comprises a CDR3 comprising the amino acid sequence set forth in SEQ ID NO: 18 or a conservative modification thereof; or
d) the α chain variable region comprises a CDR3 comprising the amino acid sequence set forth in SEQ ID NO: 41 or a conservative modification thereof, and the β chain variable region comprises a CDR3 comprising the amino acid sequence set forth in SEQ ID NO: 44 or a conservative modification thereof.

Embodiment 15. The recombinant TCR of embodiment 13 or 14, wherein:
a) the α chain variable region comprises a CDR2 comprising the amino acid sequence set forth in SEQ ID NO: 30 or a conservative modification thereof, and the β chain variable region comprises a CDR2 comprising the amino acid sequence set forth in SEQ ID NO: 32 or a conservative modification thereof;
b) the α chain variable region comprises a CDR2 comprising the amino acid sequence set forth in SEQ ID NO: 34 or a conservative modification thereof, and the β chain variable region comprises a CDR2 comprising the amino acid sequence set forth in SEQ ID NO: 36 or a conservative modification thereof;
c) the α chain variable region comprises a CDR2 comprising the amino acid sequence set forth in SEQ ID NO: 38 or a conservative modification thereof, and the β chain variable region comprises a CDR2 comprising the amino acid sequence set forth in SEQ ID NO: 32 or a conservative modification thereof; or
d) the α chain variable region comprises a CDR2 comprising the amino acid sequence set forth in SEQ ID NO: 40 or a conservative modification thereof, and the β chain variable region comprises a CDR2 comprising the amino acid sequence set forth in SEQ ID NO: 43 or a conservative modification thereof.

Embodiment 16. The recombinant TCR of any one of embodiments 13-15, wherein:
a) the α chain variable region comprises a CDR1 comprising the amino acid sequence set forth in SEQ ID NO: 29 or a conservative modification thereof, and the β chain variable region comprises a CDR1 comprising the amino acid sequence set forth in SEQ ID NO: 31 or a conservative modification thereof;
b) the α chain variable region comprises a CDR1 comprising the amino acid sequence set forth in SEQ ID NO: 33 or a conservative modification thereof, and the β chain variable region comprises a CDR1 comprising the amino acid sequence set forth in SEQ ID NO: 35 or a conservative modification thereof;
c) the α chain variable region comprises a CDR1 comprising the amino acid sequence set forth in SEQ ID NO: 37 or a conservative modification thereof, and the β chain variable region comprises a CDR1 comprising the amino acid sequence set forth in SEQ ID NO: 31 or a conservative modification thereof; or
d) the α chain variable region comprises a CDR1 comprising the amino acid sequence set forth in SEQ ID NO: 39 or a conservative modification thereof, and the β chain variable region comprises a CDR1 comprising the amino acid sequence set forth in SEQ ID NO: 42 or a conservative modification thereof.

Embodiment 17. The recombinant TCR of any one of embodiments 13-16, wherein:
a) the α chain variable region comprises a CDR1 comprising the amino acid sequence set forth in SEQ ID NO: 29 or a conservative modification thereof, a CDR2 comprising the amino acid sequence set forth in SEQ ID NO: 30 or a conservative modification thereof, and a CDR3 comprising the amino acid sequence set forth in SEQ ID NO: 9 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 10 or a conservative modification thereof;
b) the α chain variable region comprises a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 13 or a conservative modification thereof; and the β chain variable region comprises a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 14 or a conservative modification thereof;
c) the α chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 17 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 18 or a conservative modification thereof; or
d) the α chain variable region comprises a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof; and the β chain variable region comprises a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof.

Embodiment 18. The recombinant TCR of any one of embodiments 13-17, wherein:
a) the α chain variable region a CDR1 comprising the amino acid sequence set forth in SEQ ID NO: 29, a CDR2 comprising the amino acid sequence set forth in SEQ ID NO: 30, and a CDR3 comprising the amino acid sequence set forth in SEQ ID NO: 9; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 10;
b) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 13; and the β chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 14;
c) the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18; or
d) the α chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41; and the β chain variable region comprises a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44.

Embodiment 19. The recombinant TCR of any one of embodiments 13-18, wherein the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18.

Embodiment 20. The recombinant TCR of embodiment 13, wherein:
a) the α chain variable region comprises a CDR1, a CDR2, and a CDR3 of the α chain variable region having the amino acid sequence set forth in SEQ ID NO: 11; and the β chain variable region comprises a CDR1, a CDR2, and a CDR3 of the β chain variable region having the amino acid sequence set forth in SEQ ID NO: 12;
b) the α chain variable region comprises a CDR1, a CDR2, and a CDR3 of the α chain variable region having the amino acid sequence set forth in SEQ ID NO: 15; and the β chain variable region comprises a CDR1, a CDR2, and a CDR3 of the β chain variable region having the amino acid sequence set forth in SEQ ID NO: 16;
c) the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20; or
d) the α chain variable region comprises a CDR1, a CDR2, and a CDR3 of the α chain variable region having the amino acid sequence set forth in SEQ ID NO: 45; and the β chain variable region comprises a CDR1, a CDR2, and a CDR3 of the β chain variable region having the amino acid sequence set forth in SEQ ID NO: 46.

Embodiment 21. The recombinant TCR of embodiment 20, wherein the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20.

Embodiment 22. The recombinant TCR of any one of embodiments 13-21, wherein the α chain variable region comprises an amino acid sequence that is at least about 80% homologous or identical to the amino acid sequence set forth in SEQ ID NO: 11, SEQ ID NO: 15, SEQ ID NO: 19, or SEQ ID NO: 45.

Embodiment 23. The recombinant TCR of embodiment 22, wherein the α chain variable region comprises the amino acid sequence set forth in SEQ ID NO: 11, SEQ ID NO: 15, SEQ ID NO: 19, or SEQ ID NO: 45.

Embodiment 24. The recombinant TCR of embodiment 23, wherein the α chain variable region comprises the amino acid sequence set forth in SEQ ID NO: 19.

Embodiment 25. The recombinant TCR of any one of embodiments 13-24, wherein the β chain variable region comprises an amino acid sequence that is at least about 80% homologous or identical to the amino acid sequence set forth in SEQ ID NO: 12, SEQ ID NO: 16, SEQ ID NO: 20, or SEQ ID NO: 46.

Embodiment 26. The recombinant TCR of embodiment 25, wherein the β chain variable region comprises the amino acid sequence set forth in SEQ ID NO: 12, SEQ ID NO: 16, SEQ ID NO: 20, or SEQ ID NO: 46.

Embodiment 27. The recombinant TCR of embodiment 26, wherein the β chain variable region comprises the amino acid sequence set forth in SEQ ID NO: 20.

Embodiment 28. The recombinant TCR of any one of embodiments 13-27, wherein:
a) the α chain variable region comprises an amino acid sequence that is at least about 80% homologous or identical to the amino acid sequence set forth in SEQ ID NO: 11, and the β chain variable region comprises an amino acid sequence that is at least about 80% homologous or identical to the amino acid sequence set forth in SEQ ID NO: 12;
b) the α chain variable region comprises an amino acid sequence that is at least about 80% homologous or identical to the amino acid sequence set forth in SEQ ID NO: 15, and the β chain variable region comprises an amino acid sequence that is at least about 80% homologous or identical to the amino acid sequence set forth in SEQ ID NO: 16;
c) the α chain variable region comprises an amino acid sequence that is at least about 80% homologous or identical to the amino acid sequence set forth in SEQ ID NO: 19, and the β chain variable region comprises an amino acid sequence that is at least about 80% homologous or identical to the amino acid sequence set forth in SEQ ID NO: 20; or
d) the α chain variable region comprises an amino acid sequence that is at least about 80% homologous or identical to the amino acid sequence set forth in SEQ ID NO: 45, and the β chain variable region comprises an amino acid sequence that is at least about 80% homologous or identical to the amino acid sequence set forth in SEQ ID NO: 46.

Embodiment 29. The recombinant TCR of any one of embodiments 13-28, wherein:
a) the α chain variable region comprises the amino acid sequence set forth in SEQ ID NO: 11, and the β chain variable region comprises the amino acid sequence set forth in SEQ ID NO: 12;
b) the α chain variable region comprises the amino acid sequence set forth in SEQ ID NO: 15, and the β chain variable region comprises the amino acid sequence set forth in SEQ ID NO: 16;
c) the α chain variable region comprises the amino acid sequence set forth in SEQ ID NO: 19, and the β chain variable region comprises the amino acid sequence set forth in SEQ ID NO: 20; or
d) the α chain variable region comprises the amino acid sequence set forth in SEQ ID NO: 45, and the β chain variable region comprises the amino acid sequence set forth in SEQ ID NO: 46.

Embodiment 30. The recombinant TCR of embodiment 29, wherein the α chain variable region comprises the amino acid sequence set forth in SEQ ID NO: 19, and the β chain variable region comprises the amino acid sequence set forth in SEQ ID NO: 20.

Embodiment 31. The recombinant TCR of any one of embodiments 13-30, wherein the extracellular domain binds to the same EWSR1/WT1 peptide as a reference TCR or a functional fragment thereof, wherein the reference TCR or functional fragment thereof comprises an α chain variable region and a β chain variable region, wherein:
a) the α chain variable region comprises a CDR3 comprising the amino acid sequence set forth in SEQ ID NO: 9; and the β chain variable region comprises a CDR3 comprising the amino acid sequence set forth in SEQ ID NO: 10;
b) the α chain variable region comprises a CDR3 comprising the amino acid sequence set forth in SEQ ID NO: 13; and the β chain variable region comprises a CDR3 comprising the amino acid sequence set forth in SEQ ID NO: 14;
c) the α chain variable region comprises a CDR3 comprising the amino acid sequence set forth in SEQ ID NO: 17; and the β chain variable region comprises a CDR3 comprising the amino acid sequence set forth in SEQ ID NO: 18; or
d) the α chain variable region comprises the amino acid sequence set forth in SEQ ID NO: 45, and the β chain variable region comprises the amino acid sequence set forth in SEQ ID NO: 46.

Embodiment 32. The recombinant TCR of any one of embodiments 1-31, wherein the TCR is recombinantly expressed, and/or expressed from a vector.

Embodiment 33. The recombinant TCR of any one of embodiments 1-32, wherein the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 21 or SEQ ID NO: 22.

Embodiment 34. The recombinant TCR of embodiment 33, wherein the α chain constant region comprises the amino acid sequence set forth in SEQ ID NO: 21 or SEQ ID NO: 22.

Embodiment 35. The recombinant TCR of any one of embodiments 1-34, wherein the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23, SEQ ID NO: 24, SEQ ID NO: 25, or SEQ ID NO: 26.

Embodiment 36. The recombinant TCR of embodiment 35, wherein the β chain constant region comprises the amino acid sequence set forth in SEQ ID NO: 23, SEQ ID NO: 24, SEQ ID NO: 25, or SEQ ID NO: 26.

Embodiment 37. A nucleic acid encoding the T cell receptor (TCR) of any one of embodiments 1-36.

Embodiment 38. A cell comprising the TCR of any one of embodiments 1-36 or the nucleic acid of embodiment 37.

Embodiment 39. The cell of embodiment 38, wherein the cell is transduced with the TCR.

Embodiment 40. The cell of embodiment 38 or 39, wherein the TCR is constitutively expressed on the surface of the cell.

Embodiment 41. The cell of any one of embodiments 38-40, wherein the cell is an immunoresponsive cell.

Embodiment 42. The cell of any one of embodiments 38-41, wherein the cell is selected from the group consisting of a T cell, a Natural Killer (NK) cell, and a pluripotent stem cell from which a lymphoid cell may be differentiated.

Embodiment 43. The cell of embodiment 42, wherein the cell is a T cell.

Embodiment 44. The cell of embodiment 43, wherein the T cell is selected from the group consisting of a cytotoxic T lymphocyte (CTL), a regulatory T cell, a γδ T cell, a Natural Killer-T cell (NK-T), a stem cell memory T cell, a central memory T cell, and an effector memory T cell.

Embodiment 45. The cell of embodiment 44, wherein the T cell is a γδ T cell.

Embodiment 46. The cell of embodiment 44, wherein the T cell is a NK-T cell.

Embodiment 47. The cell of embodiment 42, wherein the cell is Natural Killer (NK) cell.

Embodiment 48. The cell of any one of embodiments 38-47, wherein a) the TCR is encoded by a nucleic acid integrated at a locus within the genome of the cell; or b) the nucleic acid is integrated at a locus within the genome of the cell.

Embodiment 49. The cell of embodiment 48, wherein the locus is selected from a TRAC locus, a TRBC locus, a TRDC locus, or a TRGC locus.

Embodiment 50. The cell of embodiment 48 or 49, wherein the locus is a TRAC locus or a TRBC locus.

Embodiment 51. The cell of embodiment 48, wherein the locus is selected from a PDCD1 locus, a CBLB locus, a CISH locus, and a RASA2 locus.

Embodiment 52. The cell of embodiment 48, wherein the locus is a genomic safe harbor.

Embodiment 53. The cell of any one of embodiments 38-52, wherein the cell further comprises a recombinant or exogenous co-receptor.

Embodiment 54. The cell of embodiment 53, wherein the co-receptor is a CD8 co-receptor.

Embodiment 55. The cell of embodiment 54, wherein the CD8 co-receptor comprises an α chain and a β chain.

Embodiment 56. The cell of embodiment 55, wherein the α chain comprises the amino acid sequence set forth in SEQ ID NO: 48.

Embodiment 57. The cell of embodiment 55 or 56, wherein the β chain comprises the amino acid sequence set forth in SEQ ID NO: 49.

Embodiment 58. The cell of embodiment 53, wherein the co-receptor is a CD4 co-receptor.

Embodiment 59. A composition comprising the cell of any one of embodiments 38-58.

Embodiment 60. The composition of embodiment 59, which is a pharmaceutical composition further comprising a pharmaceutically acceptable carrier.

Embodiment 61. A vector comprising the nucleic acid of embodiment 37.

Embodiment 62. The vector of embodiment 61, wherein the vector is a γ-retroviral vector.

Embodiment 63. A lipid nanoparticle comprising the nucleic acid of embodiment 37.

Embodiment 64. A method for producing a cell that binds to a EWSR1/WT1 peptide, comprising introducing into the cell the nucleic acid of embodiment 37, the vector of embodiment 61 or 62, or the lipid nanoparticle of embodiment 63.

Embodiment 65. A method of treating and/or preventing a tumor associated with EWSR1/WT1 fusion protein in a subject, comprising administering to the subject an effective amount of the cell of any one of embodiments 38-58, the composition of embodiment 59 or 60, the vector of embodiment 61 or 62, or the lipid nanoparticle of embodiment 63.

Embodiment 66. A method of reducing tumor burden in a subject having a tumor associated with EWSR1/WT1 fusion protein, comprising administering to the subject an effective amount of the cell of any one of embodiments 38-58, the composition of embodiment 59 or 60, the vector of embodiment 61 or 62, or the lipid nanoparticle of embodiment 63.

Embodiment 67. The method of embodiment 65 or 66, wherein the EWSR1/WT1 peptide comprises a junctional amino acid sequence of the fusion protein between EWS and WT1.

Embodiment 68. The method of any one of embodiments 65-67, wherein the tumor is selected from the group consisting of desmoplastic small round cell tumor (DSRCT), soft tissue sarcoma, colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

Embodiment 69. The method of embodiment 68, wherein the tumor is desmoplastic small round cell tumor (DSRCT).

Embodiment 70. The method of any one of embodiments 65-69, wherein the subject comprises an HLA-A.

Embodiment 71. The method of embodiment 70, wherein the HLA-A is an HLA-A*03 superfamily member.

Embodiment 72. The method of embodiment 71, wherein the HLA-A*03 superfamily member is selected from the group consisting of HLA-A*03, HLA-A*11, HLA-A*33, HLA-A*66, HLA-A*68, and HLA-A*74.

Embodiment 73. The method of embodiment 71 or 72, wherein the HLA-A*03 superfamily member is HLA-A*03.

Embodiment 74. The method of embodiment 73, wherein the HLA-A*03 molecule is selected from the group consisting of HLA-A*0301, HLA-A*0302, and HLA-A*0305.

Embodiment 75. The method of embodiment 71 or 72, wherein the HLA-A*03 superfamily member is HLA-A*11.

Embodiment 76. The method of embodiment 75, wherein the HLA-A*11 molecule is selected from the group consisting of HLA-A*1101, HLA-A*1102, HLA-A*1104, and HLA-A*1105.

Embodiment 77. The method of any one of embodiments 63-71, wherein the subject is a human.

Embodiment 78. The cell of any one of embodiments 38-58, the composition of embodiment 59 or 60, the vector of embodiment 61 or 62, or the lipid nanoparticle of embodiment 63 for use in treating and/or preventing a tumor associated with EWSR1/WT1 fusion protein in a subject.

Embodiment 79. The cell of any one of embodiments 38-58, the composition of embodiment 59 or 60, the vector of embodiment 61 or 62, or the lipid nanoparticle of embodiment 63 for use in reducing tumor burden in a subject having a tumor associated with EWSR1/WT1 fusion protein.

Embodiment 80. The cell, the composition, the vector, or the lipid nanoparticle for use of embodiment 78 or 79, wherein the EWSR1/WT1 peptide comprises a junctional amino acid sequence of the fusion protein between EWS and WT1.

Embodiment 81. The cell, the composition, the vector, or the lipid nanoparticle for use of any one of embodiments 78-80, wherein the tumor is selected from the group consisting of desmoplastic small round cell tumor (DSRCT), soft tissue sarcoma, colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

Embodiment 82. The cell, the composition, the vector, or the lipid nanoparticle for use of embodiment 81, wherein the tumor is desmoplastic small round cell tumor (DSRCT).

Embodiment 83. The cell, the composition, the vector, or the lipid nanoparticle for use of any one of embodiments 78-82, wherein the subject comprises an HLA-A.

Embodiment 84. The cell, the composition, the vector, or the lipid nanoparticle for use of embodiment 83, wherein the HLA-A is an HLA-A*03 superfamily member.

Embodiment 85. The cell, the composition, the vector, or the lipid nanoparticle for use of embodiment 84, wherein the HLA-A*03 superfamily member is selected from the group consisting of HLA-A*03, HLA-A*11, HLA-A*33, HLA-A*66, HLA-A*68, and HLA-A*74.

Embodiment 86. The cell, the composition, the vector, or the lipid nanoparticle for use of embodiment 84 or 85, wherein the HLA-A*03 superfamily member is HLA-A*03.

Embodiment 87. The cell, the composition, the vector, or the lipid nanoparticle for use of embodiment 86, wherein the HLA-A*03 molecule is selected from the group consisting of HLA-A*0301, HLA-A*0302, and HLA-A*0305.

Embodiment 88. The cell, the composition, the vector, or the lipid nanoparticle for use of embodiment 84 or 85, wherein the HLA-A*03 superfamily member is HLA-A*11.

Embodiment 89. The cell, the composition, the vector, or the lipid nanoparticle for use of embodiment 88, wherein the HLA-A*11 molecule is selected from the group consisting of HLA-A*1101, HLA-A*1102, HLA-A*1104, and HLA-A*1105.

Embodiment 90. The cell, the composition, the vector, or the lipid nanoparticle for use of any one of embodiments 78-89, wherein the subject is a human.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1

The presently disclosed subject matter discovered a neoantigen derived from the EWSR1/WT1 fusion protein spanning the junction of this fusion protein that is processed and presented in the context of HLA-A*03 and HLA-A*11. Together, these alleles are expressed by ˜33% of North Americans. Subsequently, T cells were identified from either HLA-A*03 or HLA-A*11 expressing healthy donors that specifically recognized the fusion-derived neoepitope. Using single cell sequencing, three T cell receptor (TCR) gene sequences that conferred recognition to the EWSR-WT1+/HLA-A*11⁺ or EWSR1-WT1+/HLA-A*03⁺ target cells were retrieved and functionally validated.

The presently disclosed subject matter provides T cell receptors (TCRs) that specifically target a fusion-derived neoepitope derived from the EWSR1/WT1 fusion protein which drives tumorigenesis of desmoplastic small round cell tumor (DSRCT). It relates to T cell receptors (TCRs) specifically targeting the EWSR1/WT1 fusion protein containing the junctional amino acid sequence which is processed and presented in the context of the HLA-A*03 and HLA-A*11 alleles. Together, these alleles are expressed by ˜33% of North Americans. The presently disclosed subject matter relates to T cell receptors (TCRs) and methods of using such TCRs and such cells for treating EWSR1/WT1 driven cancer, primarily desmoplastic small round cell tumor.

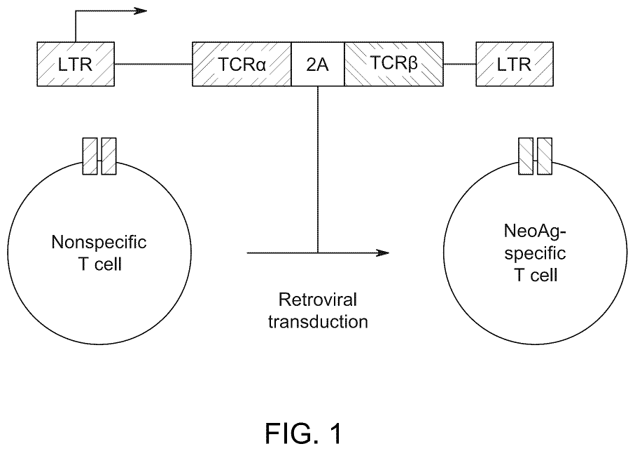

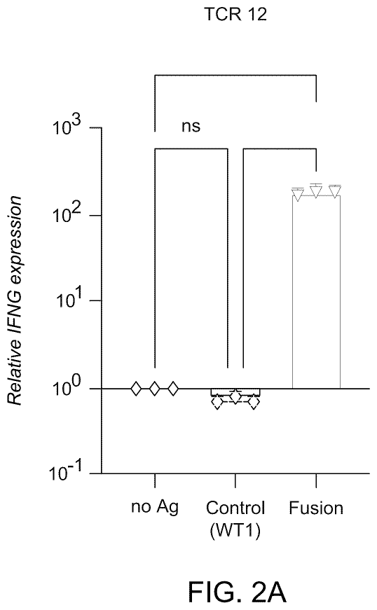

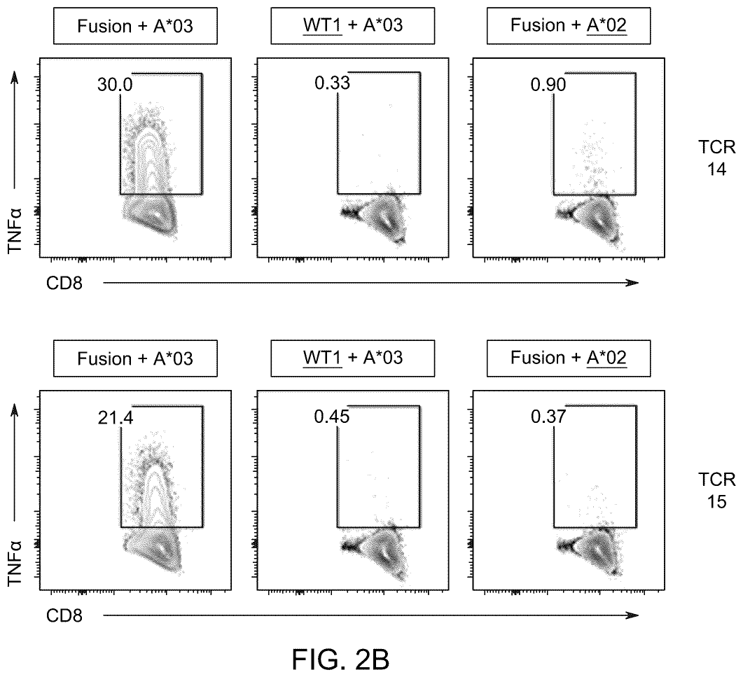

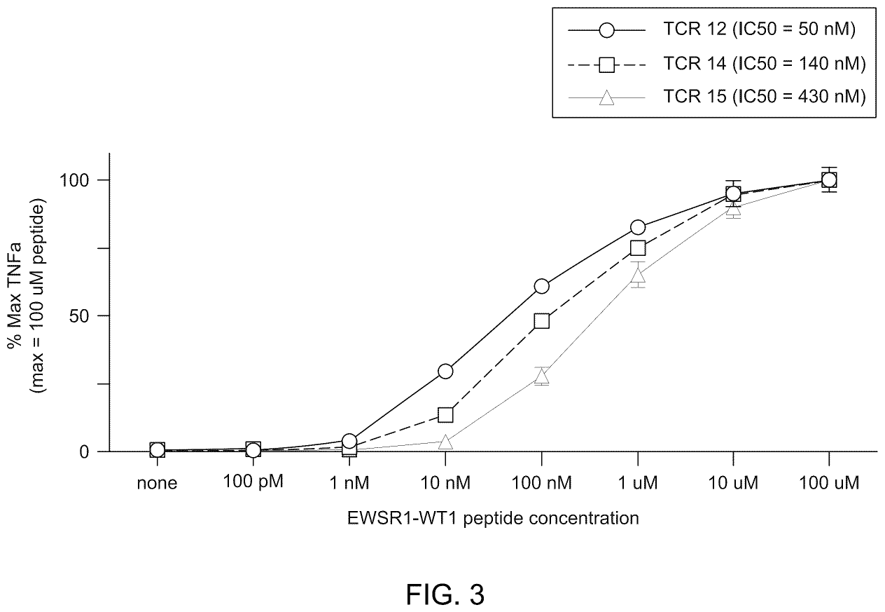

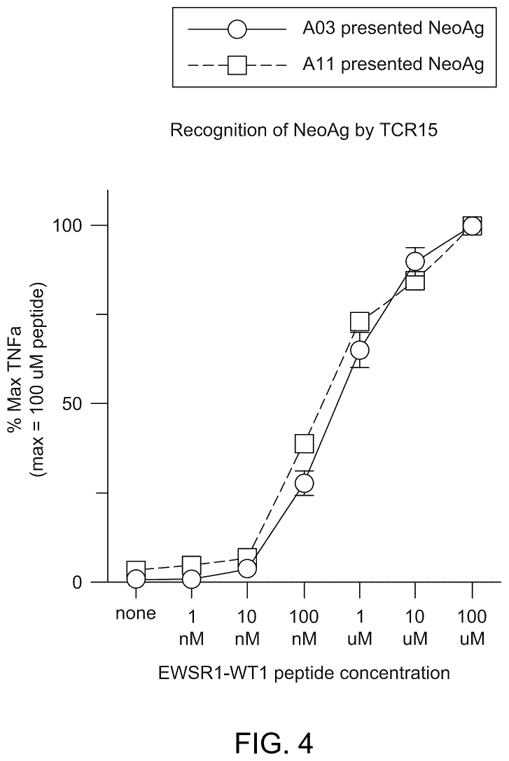

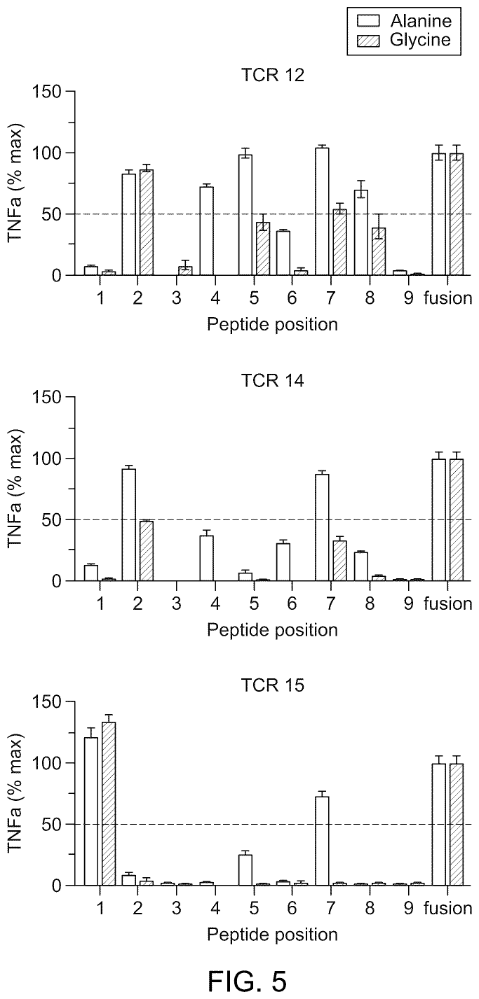

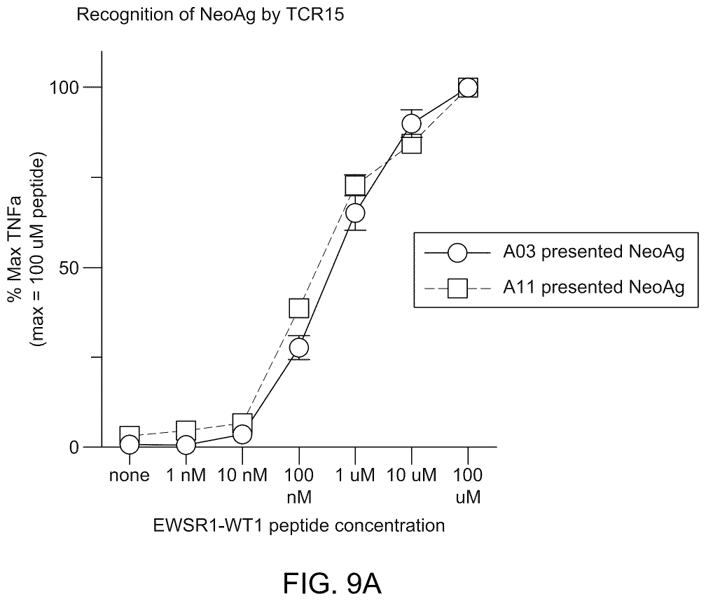

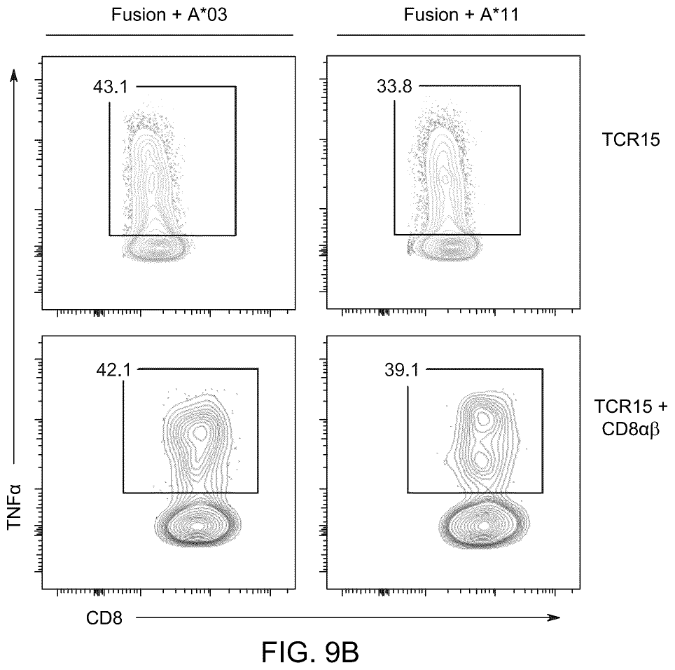

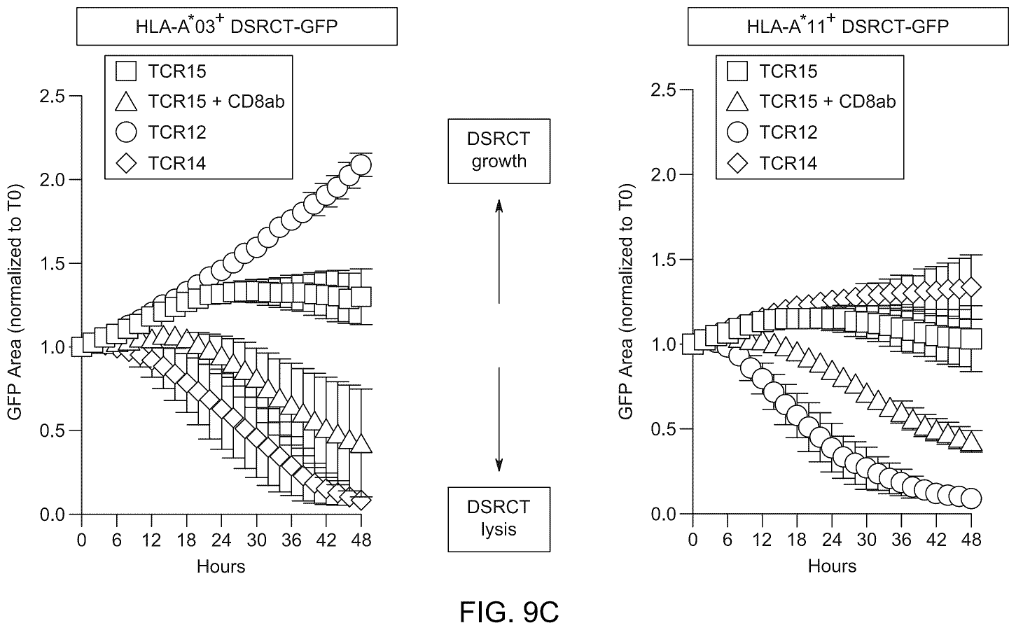

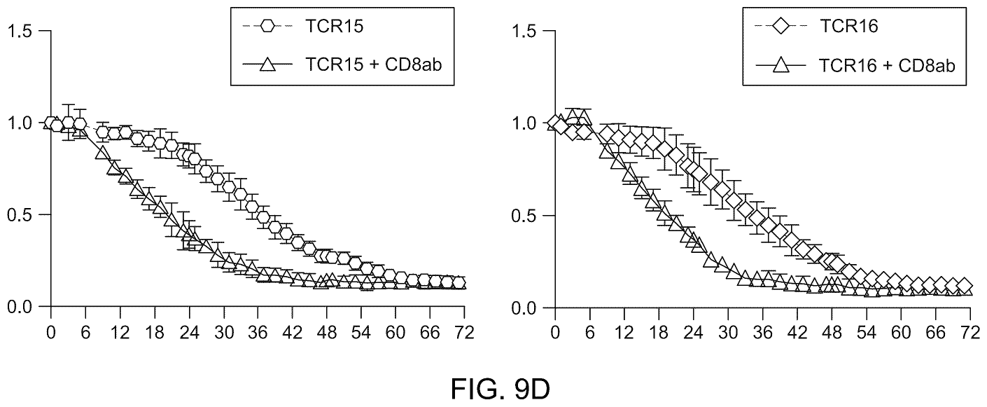

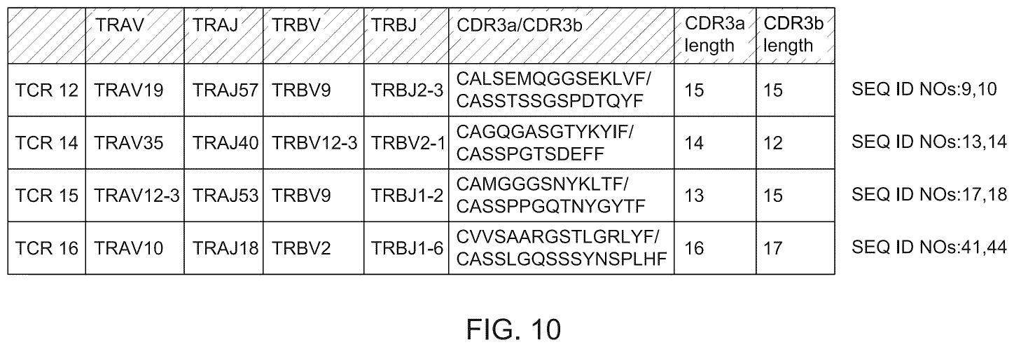

T cells from either HLA-A*03 or HLA-A*11 expressing healthy donors were used to identify T cell receptors (TCRs) recognizing the fusion-derived neoepitope. Candidate TCRs were cloned into retroviral vectors and transduced into nonspecific CD8⁺ T cells to obtain NeoAg-specific T cells as shown in FIG. 1. Single cell sequencing was used to isolate three T cell receptors designated as TCR12, TCR14, and TCR 15 that specifically recognize the EWSR1/WT1 fusion protein (See FIGS. 2A, 2B, and 3). These TCRs conferred recognition to the EWSR1/WT1⁺/HLA-A*11⁺ or EWSR1/WT1⁺/HLA-A*03⁺ target cells when transduced into nonspecific CD8⁺ T cells but not EWSR1/WT1⁻/HLA*02 mismatched target cells (FIG. 2B). Notably, as show in FIGS. 4 and 9A, TCR15 recognized both HLA-A*11⁺ and HLA-A*03⁺ target cells indicating some degree of HLA-independent EWSR1/WT1 NeoAg recognition. To further confirm these findings, Ala/Gly peptide scanning mutagenesis was performed. As shown in FIGS. 4 and 5, TCR15 was permissive to the two different HLA molecules (HLA-A03 and HLA-A11) (see FIG. 4), but not cross-reactive in general (see FIG. 5).

### Example 2

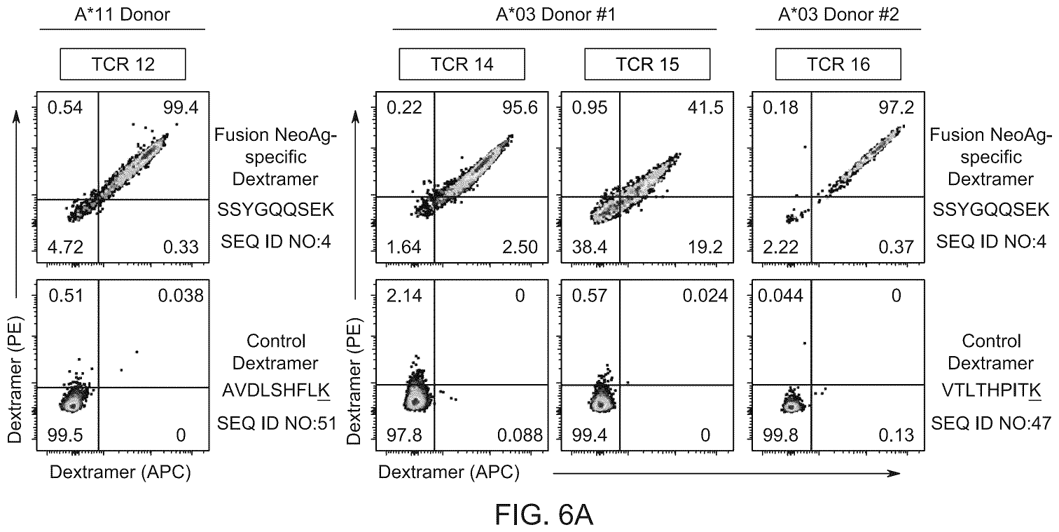

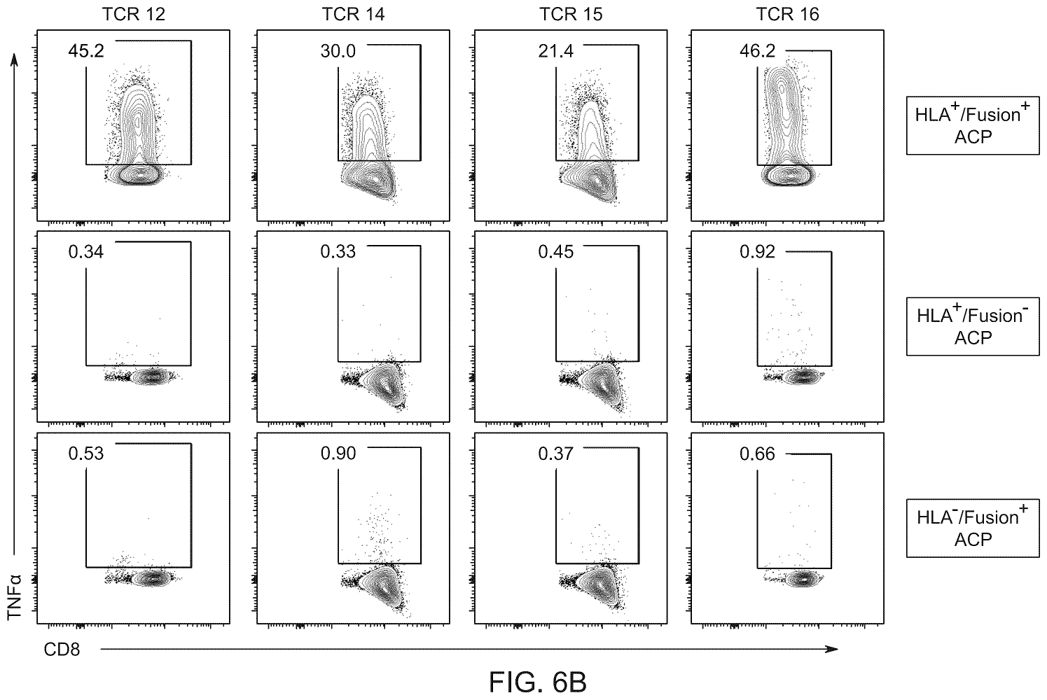

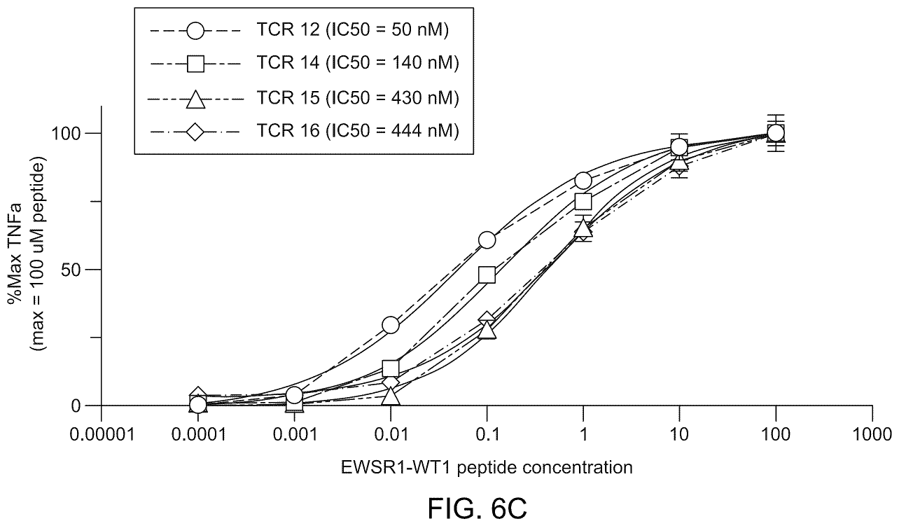

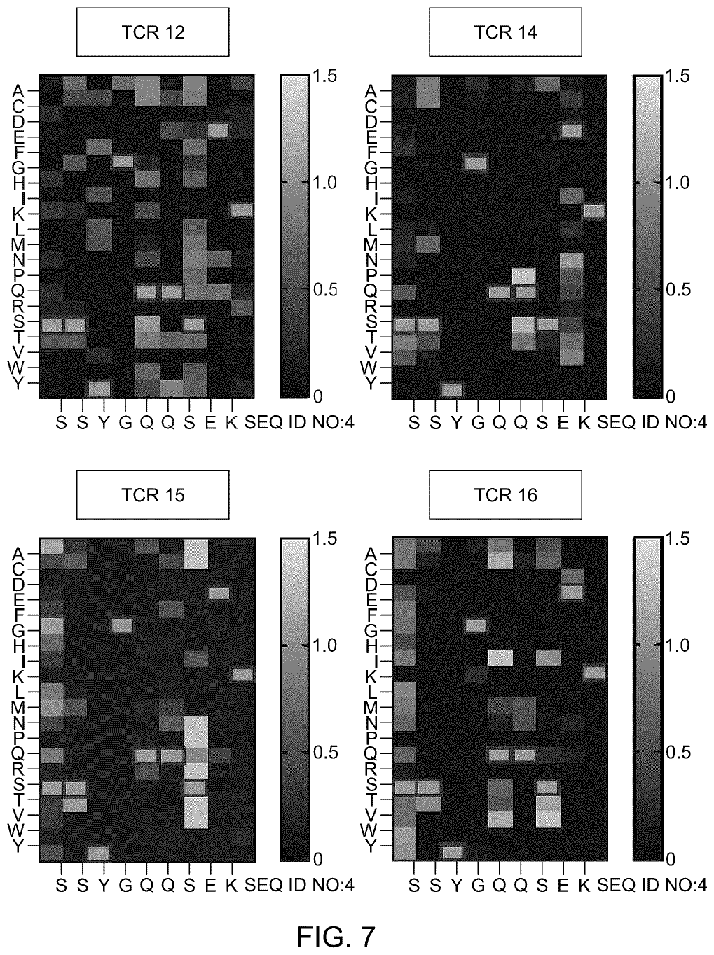

The presently disclosed TCRs (TCR12, TCR14, TCR15, and TCR16) were evaluated to determine their EWSR1/WT1 NeoAg recognition. Candidate TCRs were cloned into retroviral vectors and transduced into nonspecific CD8⁺ T cells to obtain NeoAg-specific T cells as shown in FIG. 1. As shown in FIGS. 6A-6C, the TCR conferred the ability to specifically recognize the EWSR1/WT1 NeoAg in the context of HLA as well as polyfunctional activities (e.g., TNFα production) upon binding to the EWSR1/WT1 NeoAg. Importantly, peptide substitution library experiments shown in FIG. 7 indicated the sequence specificity of the candidate TCRs.

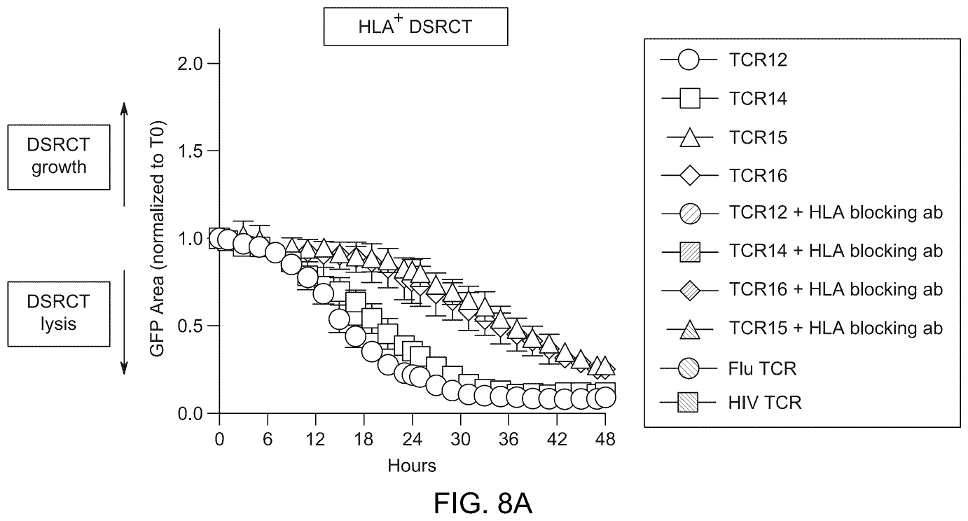

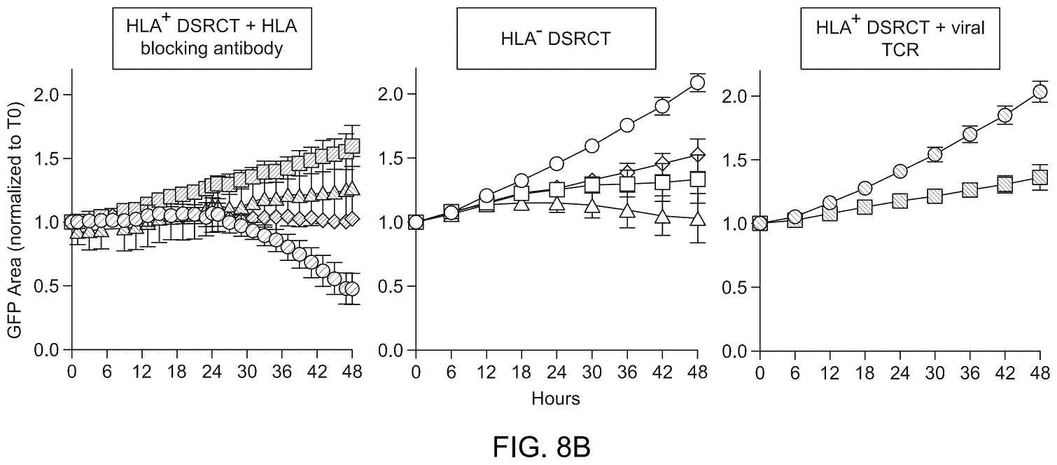

To further validate the functional properties of the TCRs disclosed herein, cell lysis experiments were performed. As shown in FIGS. 8A and 8B, T cells expressing the presently disclosed TCR had significant killing activity against DSRCT cells expressing the EWSR1/WT1 NeoAg. Notably, this effect was not observed or was attenuated when anti-HLA blocking antibodies were used or when HLA⁻DSRCT cells were used.

Since TCR15 allowed CD8⁺ T cells to recognize both HLA-A*11⁺ and HLA-A*03⁺ target cells (FIGS. 4 and 9A), the inventors of the presently disclosed subject matter determined whether expression of an exogenous CD8 receptor (CD8αβ) could enhance recognition of the target cells. As seen in FIGS. 9B-9D, CD4⁺ and CD8⁺ T cells expressing TCR15 and the exogenous CD8 receptor (CD8αβ) had increased recognition of the target cells. Comparable results were observed having CD8⁺ T cells expressing TCR16 and the exogenous CD8 receptor (CD8αβ). Overall, these data show that the addition of CD8αβ provides killing activity to CD4⁺ T cells, enhances the recognition of the target cells, and improves the functional activity (e.g., killing) of TCRs having reduced avidity.

From the foregoing description, it will be apparent that variations and modifications may be made to the invention described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. A recombinant T cell receptor (TCR) that binds to an EWSR1/WT1 peptide,
wherein the TCR comprises an extracellular domain that binds to the EWSR1/WT1 peptide,
wherein the extracellular domain comprises an α chain and a β chain, wherein the α chain comprises an α chain variable region and a chain constant region, and the β chain comprises a β chain variable region and a β chain constant region, wherein:
a) the α chain variable region comprises a CDR1 comprising the amino acid sequence set forth in SEQ ID NO: 29 or a conservative modification thereof, a CDR2 comprising the amino acid sequence set forth in SEQ ID NO: 30 or a conservative modification thereof, and a CDR3 comprising the amino acid sequence set forth in SEQ ID NO: 9 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 10 or a conservative modification thereof;
b) the α chain variable region comprises a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 13 or a conservative modification thereof; and the β chain variable region comprises a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 14 or a conservative modification thereof;
c) the α chain variable region comprises a CDR1 comprising the amino acid sequence set forth in SEQ ID NO: 37 or a conservative modification thereof, a CDR2 comprising the amino acid sequence set forth in SEQ ID NO: 38 or a conservative modification thereof, and a CDR3 comprising the amino acid sequence set forth in SEQ ID NO: 17 or a conservative modification thereof; and the β chain variable region comprises a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 18 or a conservative modification thereof; or
d) the α chain variable region comprises a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof; and the β chain variable region comprises a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof.

2. The recombinant TCR of claim 1, wherein:
a) the α chain variable region a CDR1 comprising the amino acid sequence set forth in SEQ ID NO: 29, a CDR2 comprising the amino acid sequence set forth in SEQ ID NO: 30, and a CDR3 comprising the amino acid sequence set forth in SEQ ID NO: 9; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 10;
b) the α chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 13; and the β chain variable region comprises a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 14;
c) the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18; or
d) the α chain variable region comprises a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41; and the β chain variable region comprises a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44.

3. The recombinant TCR of claim 1, wherein the α chain variable region comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 17; and the β chain variable region comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 18.

4. A recombinant T cell receptor (TCR) that binds to an EWSR1/WT1 peptide,
wherein the TCR comprises an extracellular domain that binds to the EWSR1/WT1 peptide,
wherein the extracellular domain comprises an α chain and a β chain, wherein the α chain comprises an α chain variable region and a chain constant region, and the β chain comprises a β chain variable region and a β chain constant region, wherein:
a) the α chain variable region comprises a CDR1, a CDR2, and a CDR3 of the α chain variable region having the amino acid sequence set forth in SEQ ID NO: 11; and the β chain variable region comprises a CDR1, a CDR2, and a CDR3 of the β chain variable region having the amino acid sequence set forth in SEQ ID NO: 12;
b) the α chain variable region comprises a CDR1, a CDR2, and a CDR3 of the α chain variable region having the amino acid sequence set forth in SEQ ID NO: 15; and the β chain variable region comprises a CDR1, a CDR2, and a CDR3 of the β chain variable region having the amino acid sequence set forth in SEQ ID NO: 16;
c) the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20; or
d) the α chain variable region comprises a CDR1, a CDR2, and a CDR3 of the α chain variable region having the amino acid sequence set forth in SEQ ID NO: 45; and the β chain variable region comprises a CDR1, a CDR2, and a CDR3 of the β chain variable region having the amino acid sequence set forth in SEQ ID NO: 46.

5. The recombinant TCR of claim 4, wherein the α chain variable region comprises a CDR1, a CDR2, and a CDR3 of the α chain variable region having the amino acid sequence set forth in SEQ ID NO: 19; and the β chain variable region comprises a CDR1, a CDR2, and a CDR3 of the β chain variable region having the amino acid sequence set forth in SEQ ID NO: 20.

6. The recombinant TCR of claim 1, wherein:
a) the α chain variable region comprises an amino acid sequence that is at least about 80% homologous or identical to the amino acid sequence set forth in SEQ ID NO: 11, and the β chain variable region comprises an amino acid sequence that is at least about 80% homologous or identical to the amino acid sequence set forth in SEQ ID NO: 12;
b) the α chain variable region comprises an amino acid sequence that is at least about 80% homologous or identical to the amino acid sequence set forth in SEQ ID NO: 15, and the β chain variable region comprises an amino acid sequence that is at least about 80% homologous or identical to the amino acid sequence set forth in SEQ ID NO: 16;
c) the α chain variable region comprises an amino acid sequence that is at least about 80% homologous or identical to the amino acid sequence set forth in SEQ ID NO: 19, and the β chain variable region comprises an amino acid sequence that is at least about 80% homologous or identical to the amino acid sequence set forth in SEQ ID NO: 20; or
d) the α chain variable region comprises an amino acid sequence that is at least about 80% homologous or identical to the amino acid sequence set forth in SEQ ID NO: 45, and the β chain variable region comprises an amino acid sequence that is at least about 80% homologous or identical to the amino acid sequence set forth in SEQ ID NO: 46.

7. The recombinant TCR of claim 1, wherein:
a) the α chain variable region comprises the amino acid sequence set forth in SEQ ID NO: 11, and the β chain variable region comprises the amino acid sequence set forth in SEQ ID NO: 12;
b) the α chain variable region comprises the amino acid sequence set forth in SEQ ID NO: 15, and the β chain variable region comprises the amino acid sequence set forth in SEQ ID NO: 16;
c) the α chain variable region comprises the amino acid sequence set forth in SEQ ID NO: 19, and the β chain variable region comprises the amino acid sequence set forth in SEQ ID NO: 20; or
d) the α chain variable region comprises the amino acid sequence set forth in SEQ ID NO: 45, and the β chain variable region comprises the amino acid sequence set forth in SEQ ID NO: 46.

8. The recombinant TCR of claim 7, wherein the α chain variable region comprises the amino acid sequence set forth in SEQ ID NO: 19, and the β chain variable region comprises the amino acid sequence set forth in SEQ ID NO: 20.

9. The recombinant TCR of claim 1, wherein the EWSR1/WT1 peptide comprises or consists of the amino acid sequence set forth in SEQ ID NO: 3, SEQ ID NO: 4, SEQ ID NO: 5, SEQ ID NO: 6, SEQ ID NO: 7, or SEQ ID NO: 8.

10. The recombinant TCR of claim 9, wherein the EWSR1/WT1 peptide is associated with an HLA class I complex.

11. The recombinant TCR of claim 10, wherein the HLA class I complex is selected from an HLA-A, an HLA-B, and an HLA-C.

12. The recombinant TCR of claim 11, wherein the HLA-A is an HLA-A*03 superfamily member.

13. The recombinant TCR of claim 12, wherein the HLA-A*03 superfamily member is selected from the group consisting of HLA-A*03, HLA-A*11, HLA-A*33, HLA-A*66, HLA-A*68, and HLA-A*74.

14. The recombinant TCR of claim 13, wherein (a) the HLA-A*03 molecule is selected from the group consisting of HLA-A*0301, HLA-A*0302, and HLA-A*0305, or (b) the HLA-A*11 molecule is selected from the group consisting of HLA-A*1101, HLA-A*1102, HLA-A*1104, and HLA-A*1105.

15. The recombinant TCR of claim 1, wherein (a) the α chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 21 or SEQ ID NO: 22, and/or (b) the β chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 23, SEQ ID NO: 24, SEQ ID NO: 25, or SEQ ID NO: 26.

16. The recombinant TCR of claim 15, wherein (a) the α chain constant region comprises the amino acid sequence set forth in SEQ ID NO: 21 or SEQ ID NO: 22, and/or (b) the β chain constant region comprises the amino acid sequence set forth in SEQ ID NO: 23, SEQ ID NO: 24, SEQ ID NO: 25, or SEQ ID NO: 26.

17. A nucleic acid encoding the recombinant T cell receptor (TCR) of claim 1.

18. A cell comprising the recombinant TCR of claim 1.

19. The cell of claim 18, wherein the cell is an immunoresponsive cell.

20. The cell of claim 18, wherein the cell is selected from the group consisting of a T cell, a Natural Killer (NK) cell, and a pluripotent stem cell from which a lymphoid cell may be differentiated.

21. The cell of claim 18, wherein the TCR is encoded by a nucleic acid integrated at a locus within the genome of the cell.

22. The cell of claim 21, wherein the locus is selected from a TRAC locus, a TRBC locus, a TRDC locus, a TRGC locus, a PDCD1 locus, a CBLB locus, a CISH locus, a RASA2 locus, or a genomic safe harbor.

23. The cell of claim 18, wherein the cell further comprises a recombinant or exogenous co-receptor.

24. The cell of claim 23, wherein the co-receptor is a CD8 co-receptor or a CD4 co-receptor.

25. A composition comprising the cell of claim 18.

26. The composition of claim 25, which is a pharmaceutical composition further comprising a pharmaceutically acceptable carrier.

27. A vector comprising the nucleic acid of claim 17.

28. A lipid nanoparticle comprising the nucleic acid of claim 17.

29. A method for producing a cell that binds to an EWSR1/WT1 peptide, comprising introducing into the cell the nucleic acid of claim 17.

30. A method of treating and/or preventing a tumor associated with EWSR1/WT1 fusion protein in a subject, and/or reducing tumor burden in a subject having a tumor associated with EWSR1/WT1 fusion protein, the method comprising administering to the subject an effective amount of the cell of claim 18.

31. The method of claim 30, wherein the tumor is selected from the group consisting of desmoplastic small round cell tumor (DSRCT), soft tissue sarcoma, colorectal cancer, thyroid cancer, pancreatic cancer, breast cancer, endometrial cancer, cervical cancer, anal cancer, bladder cancer, cholangiocarcinoma/bile duct cancer, lung cancer, ovarian cancer, esophageal cancer, gastric cancer, neuroendocrine tumor, head and neck squamous cell carcinoma, hepatobiliary cancer, appendiceal cancer, nonmelanoma skin cancer, salivary gland cancer, melanoma, cutaneous melanoma, germ cell tumor, thymic tumor, T-lymphoblastic leukemia/lymphoma, acute myeloid leukemia, B-cell leukemia, myeloproliferative neoplasm, histiocytosis, and multiple myeloma.

32. The method of claim 30, wherein the subject is a human.
